SEQUENCE LISTING (34 sequences)

SEQ ID NO: 1 - DNA, 1686 bp, Brassica juncea
gaccgaaccttcgatctcattctctggagaagaaagttcaatcctttttgattcaacaatggcgaagaacgtagggatattggctatggacatctatttccctcccacctgtgttcaacaggaagctttggaagctcatgatggagcaagcaaagggaaatacaccattggacttggtcaagattgtttagctttttgcactgagcttgaagatgttatctcaatgagtttcaatgcggtgacatctcttcttgaaaagtacaagattgaccccaagcaaattgggcgtcttgaagtaggcagtgagactgtcattgacaagagcaagtccatcaaaaccttcttgatgcaactctttgagaaatgtggaaacactgatgtagaaggtgttgactcgaccaatgcttgttatggtggaactgcggctttgctaaactgtgtcaattgggttgagagtaactcctgggatggacgctatggccttgtcatttgtactgacagtgcggtttatgcagaagggcccgcaaggcccactggaggagcagctgccattgctatgttgataggaccagatgctcctattgtttttgagagcaaattgagaggaagccacatggctcatgtctatgacttttacaagccaaatcttgctagcgagtacccggttgttgatggtaagctttcacagacatgctaccttatggcacttgactcttgctataaacatttatgcaacaagtttgagaaacttgagggcaaagagttctccatcaatgatgctgattactttgtcttccattctccatacaacaaacttgtgcagaaaagctttgctcgtctcttgtacaacgacttcttgagaaacgcaagctccattgatgaggctgccaaagaaaagttcactccttattcctccttgtccctcgatgagagttaccaaagccgtgatcttgaaaaggtgtcacaacaactagcaaaaacgtattatgatgctaaagtgcaaccaacgactttagtaccaaaacaagtcggtaacatgtacactgcttctctctacgcagcatttgcttccctcgtccacaacaaacacagcgatttggcgggaaagcgggtggttatgttctcatatggaagtggttcaaccgcaacaatgttctcattgcgtctctgcgagaaccagtctcctttcagcttatcaaacatcgcttctgtaatggatgttggcggtaaattgaaggctagacatgagtatgcacctgagaagtttgtggagacaatgaagctaatggaacataggtacggagcaaaggagtttgtgacgagcaaggaaggcatcttagaccttttggctccggggacttattatctgaaagaggttgattcattgtaccggagattctatggcaagaaaggagacgatggatccataaccaatggacactgattcttgaaagcataaatcacttgaaagattgtgtctactcattgctctttatgtttactttccaaaattctagacgtttttataaggttcttgttactttgatttagcgacagtttgcatgctcttgtttttatttatttgccattgattctctgaagctttttttttttctcttagtgtgtgttttaaataagcccgttaattttaggaattattaagatgttataataataaacctttaa

SEQ ID NO: 2 - DNA, 25 bp, Artificial Sequence
  Description of Artificial Sequence Synthetic 35S promoter forward primer
caatcccactatccttcgcaagacc

SEQ ID NO: 3 - DNA, 30 bp, Artificial Sequence
  Description of Artificial Sequence Synthetic BjHMGS1 3′-end cDNA reverse ML264 primer
ggatccataaccaatggacactgaggatcc

SEQ ID NO: 4 - DNA, 20 bp, Artificial Sequence
  Description of Artificial Sequence Synthetic ML915 primer
cattgctatgttgataggac

SEQ ID NO: 5 - protein, 15 aa, Artificial Sequence
  Description of Artificial Sequence Synthetic peptide
DESYQSRDLEKVSQQ

SEQ ID NO: 6 - protein, 461 aa, Brassica juncea
MAKNVGILAMDIYFPPTCVQQEALEAHDGASKGKYTIGLGQDCLAFCTELEDVISMSFNAVTSLLEKYKIDPKQIGRLEVGSETVIDKSKSIKTFLMQLFEKCGNTDVEGVDSTNACYGGTAALLNCVNWVESNSWDGRYGLVICTDSAVYAEGPARPTGGAAAIAMLIGPDAPIVFESKLRGSHMAHVYDFYKPNLASEYPVVDGKLSQTCYLMALDSCYKHLCNKFEKLEGKEFSINDADYFVFHSPYNKLVQKSFARLLYNDFLRNASSIDEAAKEKFTPYSSLSLDESYQSRDLEKVSQQLAKTYYDAKVQPTTLVPKQVGNMYTASLYAAFASLVHNKHSDLAGKRVVMFSYGSGSTATMFSLRLCENQSPFSLSNIASVMDVGGKLKARHEYAPEKFVETMKLMEHRYGAKEFVTSKEGILDLLAPGTYYLKEVDSLYRRFYGKKGDDGSITNGH

SEQ ID NO: 7 - DNA, 30 bp, Artificial Sequence
  Description of Artificial Sequence Synthetic ML276 primer
ggatccatggcgaagaacgtagggatattg

SEQ ID NO: 8 - DNA, 22 bp, Artificial Sequence
  Description of Artificial Sequence Synthetic ML860 primer
ggagactggttctcgcagagac

SEQ ID NO: 9 - DNA, 23 bp, Artificial Sequence
  Description of Artificial Sequence Synthetic ML1046 primer
ccataattacaccagcagtgtcc

SEQ ID NO: 10 - DNA, 24 bp, Artificial Sequence
  Description of Artificial Sequence Synthetic ML1047 primer
caactgtgccaacctcaatagaag

SEQ ID NO: 11 - DNA, 20 bp, Artificial Sequence
  Description of Artificial Sequence Synthetic NtHMGR1-specific ML1879 primer
ttggcatcggatttgttcag

SEQ ID NO: 12 - DNA, 19 bp, Artificial Sequence
  Description of Artificial Sequence Synthetic NtHMGR1-specific ML 1880 primer
ggcggctatcttcctcaat

SEQ ID NO: 13 - DNA, 20 bp, Artificial Sequence
  Description of Artificial Sequence Synthetic NtHMGR2-specific ML1881 primer
agcaggtggcgtgagaaaat

SEQ ID NO: 14 - DNA, 20 bp, Artificial Sequence
  Description of Artificial Sequence Synthetic NtHMGR2-specific ML1882 primer
cgaacgactgaacaaatccg

SEQ ID NO: 15 - DNA, 21 bp, Artificial Sequence
  Description of Artificial Sequence Synthetic NtIPI1-specific ML1885 primer
atcgggtttgttcagtcgttc

SEQ ID NO: 16 - DNA, 21 bp, Artificial Sequence
  Description of Artificial Sequence Synthetic NtIPI1-specific ML1886 primer
gcaggtccacgacgactatct

SEQ ID NO: 17 - DNA, 22 bp, Artificial Sequence
  Description of Artificial Sequence Synthetic NtIPI2-specific ML1887 primer
attgaggagaatgctcttggtg

SEQ ID NO: 18 - DNA, 22 bp, Artificial Sequence
  Description of Artificial Sequence Synthetic NtIPI2-specific ML1888 primer
ctggtcaactgggacatcttca

SEQ ID NO: 19 - DNA, 20 bp, Artificial Sequence
  Description of Artificial Sequence Synthetic NtFPPS-specific ML1889 primer
cttctccgcaaccacatcac

SEQ ID NO: 20 - DNA, 21 bp, Artificial Sequence
  Description of Artificial Sequence Synthetic NtFPPS-specific ML1890 primer
gaggcagtctggaactcaacc

SEQ ID NO: 21 - DNA, 22 bp, Artificial Sequence
  Description of Artificial Sequence Synthetic NtSQS-specific ML1678 primer
aggaggtggaaacaactgatga

SEQ ID NO: 22 - DNA, 20 bp, Artificial Sequence
  Description of Artificial Sequence Synthetic NtSQS-specific ML1679 primer
agaacatacggcacttgggt

SEQ ID NO: 23 - DNA, 20 bp, Artificial Sequence
  Description of Artificial Sequence Synthetic NtGGPPS1-specific ML1891 primer
gaccaatccaacgctatgcc

SEQ ID NO: 24 - DNA, 20 bp, Artificial Sequence
  Description of Artificial Sequence Synthetic NtGGPPS1-specific ML1892 primer
ttatggttcgtcggctttcc

SEQ ID NO: 25 - DNA, 22 bp, Artificial Sequence
  Description of Artificial Sequence Synthetic NtSMT1-2-specific ML1893 primer
tccaaataacgaagagcacgaa

SEQ ID NO: 26 - DNA, 19 bp, Artificial Sequence
  Description of Artificial Sequence Synthetic NtSMT1-2-specific ML1894 primer
caggcgaatcctcagcaag

SEQ ID NO: 27 - DNA, 19 bp, Artificial Sequence
  Description of Artificial Sequence Synthetic NtSMT2-1-specific ML1895 primer
aaaccgccgataaagtccc

SEQ ID NO: 28 - DNA, 22 bp, Artificial Sequence
  Description of Artificial Sequence Synthetic NtSMT2-1-specific ML1896 primer
tctacagccatttcttcgtgaa

SEQ ID NO: 29 - DNA, 20 bp, Artificial Sequence
  Description of Artificial Sequence Synthetic NtSMT2-2-specific ML1897 primer
atccacggtattgaaagggg

SEQ ID NO: 30 - DNA, 19 bp, Artificial Sequence
  Description of Artificial Sequence Synthetic NtSMT2-2-specific ML1898 primer
tttcagcctcgtccaccac

SEQ ID NO: 31 - DNA, 22 bp, Artificial Sequence
  Description of Artificial Sequence Synthetic NtCYP85A1-specific ML1899 primer
aagaaaagccttcctcctggta

SEQ ID NO: 32 - DNA, 22 bp, Artificial Sequence
  Description of Artificial Sequence Synthetic NtCYP85A1-specific ML1900 primer
aaaactcccaaatctggctctt

SEQ ID NO: 33 - DNA, 23 bp, Artificial Sequence
  Description of Artificial Sequence Synthetic NtACTIN-specific ML1951 primer
tcacagaagctcctcctaatcca

SEQ ID NO: 34 - DNA, 22 bp, Artificial Sequence
  Description of Artificial Sequence Synthetic NtACTIN-specific ML1952 primer
gagggaaagaacagcctgaatg

(19) United States
(12) Patent Application Publication
(10) Pub. No.: US 2014/0380524 A1
(43) Pub. Date: Dec. 25, 2014

(54) METHODS OF USING 3-HYDROXY-3-METHYLGLUTARYL-COA SYNTHASE TO ENHANCE GROWTH AND/OR SEED YIELD OF GENETICALLY MODIFIED PLANTS

(71) Applicant: The University of Hong Kong, Hong Kong (CN)

(72) Inventors: Mee Len Chye, Hong Kong (CN); Pan Liao, Hong Kong (CN); Hui Wang, Hong Kong (CN); Mingfu Wang, Hong Kong (CN)

(21) Appl. No.: 14/260,561

(22) Filed: Apr. 24, 2014

Related U.S. Application Data
(60) Provisional application No. 61/836,739, filed on Jun. 19, 2013.

(51) Int. Cl. C12N 15/82

(52) U.S. Cl. CPC C12N 15/8261; C12N 15/8209; C12N 15/8222
USPC 800/290; 800/317; 800/317.3; 800/317.4; 800/317.2; 800/317.1; 800/287

(57) ABSTRACT

Provided herein is a transgenic plant, seed, or progeny, genetically engineered to overexpress one or more exogenous 3-hydroxy-3-methylglutaryl-CoA synthase 1 (HMGS1) in an amount effective to enhance growth and/or seed yield relative to a control plant. Also provided are methods of enhancing plant growth and/or seed yield by genetically engineering a plant to overexpress one or more exogenous HMGS1 in an amount effective to enhance growth and/or seed yield relative to a control plant. The plant belongs to the Solanaceae family, and the one or more exogenous HMGS1 comprise an amino acid sequence at least 77% identical to Brassica juncea HMGS1 as set forth in SEQ ID NO:6. Further provided are methods of screening for a functional variant of Brassica juncea HMGS1.

### CROSS REFERENCE TO RELATED APPLICATIONS

This application claims priority to U.S. Provisional Application Ser. No. 61/836,739 filed Jun. 19, 2013, which is incorporated herein by reference in its entirety.

### INCORPORATION OF SEQUENCE LISTING

The Sequence Listing contained in the file named “56720-130938_SL.txt”, which is 14,640 bytes in size (measured in operating system MS-Windows) and was created on Apr. 16, 2014, is contemporaneously filed by electronic submission (using the United States Patent Office EFS-Web filing system) and is incorporated herein by reference in its entirety.

### FIELD OF THE INVENTION

The present invention generally relates to the field of plant engineering. In particular, the present invention relates to genetically engineered plants that overexpress one or more exogenous 3-hydroxy-3-methylglutaryl-CoA synthase 1 (HMGS1) in an amount effective to enhance growth and/or seed yield as well as methods of enhancing growth and/or seed yield of genetically modified plants.

### BACKGROUND OF THE INVENTION

It is desirable in modern agriculture to improve growth and/or seed yield because seed grains represent an important source of food (Jiao et al., Nat. Genet., 42:541-544, 2010). To this end, the key genes that boost seed yield must first be identified. The enzymes 3-hydroxy-3-methylglutaryl-CoA synthase (HMGS) and 3-hydroxy-3-methylglutaryl-CoA reductase (HMGR) are involved in the mevalonate (MVA) pathway (Lynen et al., Biochem. Z. 330:269-295, 1958; Ferguson Jr. and Rudney, J. Biol. Chem. 234:1072-1075, 1959; Rudney and Ferguson Jr., J. Biol. Chem. 234:1076-1080, 1959; Lynen, Pure Appl. Chem. 14:137-167, 1967; Stewart and Rudney, J. Biol. Chem. 241:1222-1225, 1966; Balasubramanlam et al., Proc. Natl. Acad. Sci. USA 74:1421-1425, 1977). Besides HMGR, HMGS is also a key enzyme in cholesterol synthesis in the mammals as well as in plants (Kimberly et al., J. Biol. Chem. 273:1349-1356, 1998; Alex et al., Plant J. 22:415-426, 2000; Wang et al., Plant Biotechnol. J. 10:31-42, 2012). In plants, studies focused initially on HMGR, and interest in HMGS occurred later (Bach, Lipids, 21:82-88, 1986; Alex et al., Plant J. 22:415-426, 2000; Ishiguro et al., Plant Cell Physiol. 51:896-911, 2010; Hemmerlin et al., Prog. Lipid. Res. 51:95-148, 2012). Four genes (BjHMGS1-BjHMGS4) encode HMGS in Brassica juncea (Alex et al., Plant J. 22:415-426, 2000). Investigations on BjHMGS1 revealed that it is localized to the cytosol and the expression of its recombinant protein in Escherichia coli led to the elucidation of its crystal structure (Nagegowda et al., Biochem. J. 383:517-527, 2004; Nagegowda et al., Planta 221:844-856, 2005; Pojer et al., Proc. Natl. Acad. Sci. USA 103:11491-11496, 2006). Analysis of the Arabidopsis hmgs mutant revealed that HMGS is involved in tapetal development and affects the fertility of pollen grains (Ishiguro et al., Plant Cell Physiol. 51:896-911, 2010). Overexpression of wild-type and mutant BjHMGS1 (H188N, S359A and H188N/S359A) has been recently investigated in transgenic Arabidopsis, which showed that BjHMGS1 overexpression in transgenic Arabidopsis up-regulated several genes in sterol biosynthesis including HMGR, SMT2, DWF1, CYP710A1 and BR6OX2, culminating in an increased sterol content by about 11.3 to 26.8% as well as in enhanced stress tolerance such as Botrytis cinerea resistance and H₂O₂ tolerance (Wang et al., Plant Biotechnol. J. 10:31-42, 2012). However, the same study showed that BjHMGS1 overexpression in transgenic Arabidopsis did not lead to any obvious phenotype change in plant growth or an increase in seed yield. Only more rapid seed germination was apparent when BjHMGS1 was overexpressed in Arabidopsis.

There is a need for genetically modified plants with enhanced growth and/or seed yield (e.g., increased pod size and seed number) and a method for producing the same. The present invention fulfills such need.

### SUMMARY OF THE INVENTION

Transgenic plants with enhanced growth and/or seed yield are provided herein. Plant parts, including but not limited to fruits, leaves, tubers, seeds, flowers, stems, roots, and other anatomical parts, wherein HMGS1 and its mutant derivatives (H188N, S359A and H188N/S359A) are expressed, are also provided herein. Moreover, methods of enhancing plant growth and/or seed yield as well as methods of screening for a functional variant of Brassica juncea HMGS1 are provided herein.

In one embodiment, there is provided a transgenic plant/seed/progeny genetically engineered to overexpress one or more exogenous HMGS1 in an amount effective to enhance growth and/or seed yield relative to a control plant. The transgenic plant belongs to the Solanaceae family, and the one or more exogenous HMGS1 comprises an amino acid sequence at least 77% identical to SEQ ID NO:6.

In another embodiment, there is provided a method of enhancing plant growth and/or seed yield. Such method comprises genetically engineering a plant to overexpress one or more exogenous HMGS1 in an amount effective to enhance growth and/or seed yield relative to a control plant. The one or more exogenous HMGS1 comprise an amino acid sequence at least 77% identical to SEQ ID NO:6.

In yet another embodiment, there is provided a method of screening for a functional variant of Brassica juncea HMGS1 which comprises the amino acid sequence as set forth in SEQ ID NO:6. Such method comprises the steps of obtaining a plant cell genetically modified to express a candidate variant; regenerating a plant from the plant cell; and determining whether the plant exhibits an increase in growth and/or seed yield, thereby determining whether the candidate variant is a functional equivalent of the Brassica juncea HMGS1.

### BRIEF DESCRIPTION OF THE DRAWINGS

FIG. 1 shows the BjHMGS1 transformation construct and resultant polymerase chain reaction (PCR) analysis on transgenic tobacco plants. Panel (a) shows the schematic map of transformation vector indicating primer locations. BjHMGS1 wild-type and mutant inserts were derived from plasmids pBj132 (H188N/S359A), pBj134 (wtBjHMGS1), pBj136 (S359A) and pBj137 (H188N). CaMV35S: Cauliflower Mosaic Virus 35S promoter; NOSpro: nopaline synthase (NOS) promoter; NOSter: NOS terminator; NPTII: neomycin phosphotransferase II gene encoding resistance to kanamycin; RB: right border of T-DNA; LB: left border of T-DNA. 35S: 35S promoter 3′-end forward primer; ML264: BjHMGS1-specific 3′-end reverse primer. Panel (b) shows agarose gel analysis, illustrating the expected 1.65-kb BjHMGS1 cDNA band from transgenic tobacco following PCR using primer pair 35S/ML264; representative lines are shown here. OE-wtBjHMGS1 (lanes 1-3); OE-H188N (lanes 4-6); OE-S359A (lanes 7-9); OE-H188N/S359A (lanes 10-12); positive PCR control (lane 13, PCR template is plasmid pBj134); blank (lane 14, no DNA in PCR reaction).

FIG. 2 shows molecular analysis of representative transgenic tobacco HMGS-OEs. Panel (a) shows the Western blot analysis using antibodies against BjHMGS1 to verify the expression of BjHMGS1 (52.4-kDa) in representative vector-transformed control (pSa13) and HMGS-OEs (OE-wtBjHMGS1, OE-H188N, OE-S359A and OE-H188N/S359A) and corresponding Coomassie Blue-stained gel of total protein (20 μg per well). Three independent lines per construct were tested. Panel (b) shows the Northern blot analysis of BjHMGS1 and endogenous HMGR mRNAs in representative vector-transformed control (pSa13) and HMGS-OEs. Expected 1.7-kb BjHMGS1 band and 2.5-kb tobacco HMGR band are arrowed. Bottom gels show rRNA loaded per lane. Two independent lines per construct are shown.

FIG. 3 shows the sterol content in tobacco HMGS-OE leaves and seedlings. Sterol content including campesterol, stigmasterol, sitosterol, and total sterol of 20-day-old seedlings grown on Petri plates and of leaves from 60-day-old potted plants grown in soil are shown. Values are means±SD (n=5); H, higher than vector-transformed control; *, P<0.05; **, P<0.01 by Student's t-test. DW: dry weight; S: seedlings; L: leaves. Bars represent SD.

FIG. 4 shows the comparison in root length and dry weight between tobacco HMGS-OE seedlings and vector (pSa13)-transformed control. Panel (a) shows seedlings 14-day post germination. Bar, 1 cm. Panel (b) shows dry weight of 14-day-old seedlings, suggesting that tobacco HMGS-OEs possess higher mass than the vector-transformed control. Values are mean±SD (n=30); Bars are SD; **, P<0.01. Panel (c) shows root length measurements of 14-day-old seedlings, suggesting that tobacco HMGS-OE roots grow faster than vector-transformed control. Values are mean±SD (n=30); Bars are SD; **, P<0.01. pSa13, vector-transformed control; OE plants are labeled wt-BjHMGS1, H188N, S359A and H188N/S359A.

FIG. 5 shows the comparison in growth between tobacco HMGS-OEs and vector-transformed control. Panel (a) shows representative greenhouse-grown plants photographed 80 days after germination. Bar=10 cm. Panel (b) shows representative greenhouse-grown plants photographed 136 days after germination. Bar=10 cm. Panel (c) shows statistical analysis on height of transgenic plants at two different growth stages, 80 days and 136 days. Values are means±SD (n=30); Bars are SD; H, higher than control; **, P<0.01 by Student's t-test. pSa13, vector-transformed control; OE plants are labeled wt-BjHMGS1, H188N, S359A and H188N/S359A.

FIG. 6 shows the comparison in plant growth between greenhouse grown HMGS-OE and vector-transformed control grown in greenhouse. Panel (a) shows representative plants photographed 98 days after germination with differences in growth between HMGS-OE tobacco plants and vector-transformed control. Bar=10 cm. Panel (b) shows representative tobacco leaves photographed 98 days after germination with differences in growth between HMGS-OE tobacco plants and vector-transformed plants. Bar=10 cm. Panel (c) shows the analyses on height of 98 days old transgenic plants. Panel (d) shows the analysis on fresh weight of bottom four leaves from a 98-day-old transgenic plant. Panel (e) shows the analysis on the length of bottom four leaves from a 98-day-old transgenic plant. Panel (f) shows the analyses on width of bottom four leaves from a 98-day-old transgenic plant. Values are mean±SD (n=6); Bars are SD; **, P<0.01; *, P<0.05; ** and *, significantly higher than control, by Student's t-test. Vector-transformed control is labeled with “pSa13”, three independent lines of wt-BjHMGS1 plants are labeled “401”, “402”, and “404” and three independent lines of S359A plants are labeled “602”, “603”, and “606”.

FIG. 7 shows tobacco HMGS-OEs with increased seed yield. Panel (a) shows the phenotype of tobacco pods. pSa13: vector-transformed control; “401” and “402”: two independent lines of wt-BjHMGS1; “603” and “606”: two independent lines of S359A. Scale bar=1 cm. Panel (b) shows the total dry weight of 30 tobacco pods. Panel (c) shows the average dry weight per pod. Panel (d) shows the total dry weight of seeds of 30 pods. Panel (e) shows the comparison in dry weight of 100 seeds between control and the tobacco HMGS-OEs. Thirty repeats were measured for each line. Results are the average dry weight per 100 tobacco seeds of 30 repeats for each line. Panel (f) shows the total seed number in 30 pods. Panel (g) shows the average seed number per pod. Values are mean±SD, n=30; **, P<0.01; *, P<0.05 by Student's t-test.

FIG. 8 shows the expression of HMGS downstream genes in 20-day-old vector-transformed control (pSa13) tobacco seedlings by qRT-PCR. Three independent lines of wt-BjHMGS1 tobacco seedlings (401, 402, and 404), and three independent lines of S359A tobacco seedlings (602, 603, and 606) were studied. Total RNA was extracted from 20-day-old tobacco seedlings of vector-transformed control (pSa13), wt-BjHMGS1 (401, 402, and 404) and S359A (602, 603, and 606). H: value higher than the control (P<0.05); L: value lower than the control (P<0.05). Values are means±SD (n=3). Nicotiana tabacum 3-hydroxy-3-methylglutaryl-CoA reductase (NtHMGR1 and NtHMGR2), isopentenyl-diphosphate delta-isomerase (NtIPI1 and NtIPI2), famesyl diphosphate synthase (NtFPPS), squalene synthase (NtSQS), geranylgeranyl diphosphate synthase (NtGGPPS1), sterol methyltransferases (NtSMT1-2, NtSMT2-1 and NtSMT2-2) and cytochrome P450 monooxygenase (NtCYP85A1) were analyzed.

FIG. 9 shows the expression of HMGS downstream genes in fully-opened vector-transformed control (pSa13) tobacco flowers. Three independent lines of wt-BjHMGS1 tobacco flowers (401, 402, and 404), and three independent lines of S359A tobacco flowers (602, 603, and 606) by qRT-PCR were studied. Total RNA was extracted from 3-week-old tobacco seedlings of vector-transformed control (pSa13), wt-BjHMGS1 (401, 402, and 404) and S359A (602, 603, and 606). H: value higher than the control (P<0.05); L: value lower than the control (P<0.05). Values are means±SD (n=3).

FIG. 10 shows the BjHMGS1 constructs used in tomato transformation and PCR analysis on resultant transgenic tomato lines. Panel (a) shows the schematic map of transformation vector indicating primer location. BjHMGS1 wild-type and mutant inserts were derived from pBj134 (wtBjHMGS1) and pBj136 (S359A), respectively (Wang et al., Plant Biotechnol J 10: 31-42, 2012). CaMV35S: Cauliflower Mosaic Virus 35S promoter; NOSpro: nopaline synthase (NOS) promoter; NOSter: NOS terminator; NPTII: gene encoding neomycin phosphotransferase II conferring resistance to kanamycin; RB: right border of T-DNA; LB: left border of T-DNA. 35S: 35S promoter 3′-end forward primer; ML860: BjHMGS1-specific 3′-end reverse primer. Panel (b) shows agarose gel analysis, illustrating the expected 1.4-kb BjHMGS1 cDNA band (arrowed) from wild-type BjHMGS1 transgenic tomato following PCR using primer pair 35S/ML860. Lane 1, 1 kb marker; lane 2, positive control (plasmid of pBj134); lane 3, negative control (plasmid of vector pSa13); lane 4, pBj134-1 (401); lane 5, pBj134-3 (403); lane 6, pBj134-5 (405); lane 7, pBj134-6 (406); lane 8, pBj134-21 (421); lane 9, pBj134-23 (423); lane 10, pBj134-30 (430); lane 11, pBj134-42 (442); lane 12, pBj134-45 (445). Plasmid pBj134 is a pSa13 derivative containing the 35S::wild-type BjHMGS1 (wtBjHMGS1) fusion (Wang et al., Plant Biotechnol. J. 10:31-42, 2012). Panel (c) shows agarose gel analysis, illustrating the expected 1.4-kb BjHMGS1 cDNA band (arrowed) from mutant BjHMGS1 (S359A) transgenic tomato following PCR using primer pair 35S/ML860. Lane 1, 1 kb marker; lane 2, positive control (plasmid of pBj134); lane 3, negative control (plasmid of vector pSa13); lane 4, pBj136-5 (605); lane 5, pBj136-7 (607); lane 6, pBj136-8 (608); lane 7, pBj136-12 (612); lane 8, pBj136-13 (613); lane 9, pBj136-14 (614); lane 10, pBj136-15 (615); lane 11, pBj136-22 (622); lane 12, pBj136-25 (625). Plasmid pBj136 is a pSa13 derivative containing the 35S::mutant BjHMGS1 (S359A) fusion (Wang et al., Plant Biotechnol. J. 10:31-42, 2012).

FIG. 11 shows molecular analysis of representative transgenic tomato HMGS-OEs. Panel (a) shows the Western blot analysis using antibodies against BjHMGS1 to verify the expression of BjHMGS1 (52.4-kDa) in representative vector (pSa13)-transformed control and wild-type HMGS-OEs (OE-wtBjHMGS1) and corresponding Coomassie Blue-stained gel of total protein (20 μg per well) loaded in a 12% SDS-PAGE gel. The cross-reacting 52.4-kDa BjHMGS1 band (arrowhead) is shown in the positive control and cross-reacting tomato lines. Lane 1, positive control (tobacco BjHMGS1 OE line “402”); lane 2, vector (pSa13)-transformed control; lane 3, pBj134-6 (406); lane 4, pBj134-10 (410); lane 5, pBj134-13 (413); lane 6, pBj134-14 (414); lane 7, pBj134-15 (415); lane 8, pBj134-27 (427); lane 9, pBj134-28 (428); lane 10, pBj134-30 (430); lane 11, pBj134-39 (439); lane 12, pBj134-42 (442); lane 13, pBj134-44 (444); lane 14, pBj134-45 (445). The two independent lines of OE-wtBjHMGS1 plants “430” and “445” (lanes 10 and 14) used in further tests are underlined. Plasmid pBj134 is a pSa13 derivative containing the 35S::wild-type BjHMGS1 (wtBjHMGS1) fusion (Wang et al., Plant Biotechnol. J. 10:31-42, 2012). Panel (b) shows the Western blot analysis using antibodies against BjHMGS1 to verify the expression of BjHMGS1 (52.4-kDa) in representative vector (pSa13)-transformed control and mutant HMGS-OEs (OE-S359A) and corresponding Coomassie Blue-stained gel of total protein (20 μg per well) loaded in a 12% SDS-PAGE gel. The cross-reacting 52.4-kDa BjHMGS1 band (arrowhead) is shown in the positive control and cross-reacting tomato lines. Lane 1, positive control (tobacco BjHMGS1 OE line “402”); lane 2, vector (pSa13)-transformed control; lane 3, pBj136-5 (605); lane 4, pBj136-13 (613); lane 5, pBj136-15 (615); lane 6, pBj136-19 (619); lane 7, pBj136-20 (620); lane 8, pBj136-22 (622); lane 9, pBj136-23 (623); lane 10, pBj136-24 (624); lane 11, pBj136-25 (625); lane 12, pBj136-31 (631); lane 13, pBj136-35 (635). The two independent lines of OE-S359A plants “622” and “625” (lanes 8 and 11) used in further tests are underlined. Plasmid pBj136 is a pSa13 derivative containing the 35S::mutant BjHMGS1 (S359A) fusion (Wang et al., Plant Biotechnol. J. 10:31-42, 2012).

FIG. 12 shows Southern blot analysis on representative transgenic tomato plants. Panel (a) shows the schematic map of transformation vector indicating EcoRI (E) sites. BjHMGS1 wild-type and mutant inserts were derived from plasmids pBj134 (wtBjHMGS1) and pBj136 (S359A). CaMV35S: Cauliflower Mosaic Virus 35S promoter; NOSpro: nopaline synthase (NOS) promoter; NOSter: NOS terminator; NPTII: gene encoding neomycin phosphotransferase II conferring resistance to kanamycin; RB: right border of T-DNA; LB: left border of T-DNA. Dotted lines denote position of nucleotide on vector. Panel (b) shows the Southern blot analysis of genomic DNA digested by restrictive endonuclease EcoRI and probed with digoxigenin-labeled BjHMGS1 full-length cDNA in representative blots. The hybridizing bands are expected to be longer than 4.8 kb (see map in panel (a)). Lane 1, pBj134-30 (430); lane 2, pBj136-15 (615); lane 3, pBj136-17 (617); lane 4, pBj136-19 (619); lane 5, pBj136-25 (625); lane 6, vector (pSa13)-transformed control; lane 7, pBj136-15 (615); lane 8, pBj136-17 (617); lane 9, pBj136-19 (619); lane 10, pBj136-21 (621); lane 11, pBj136-23 (623); lane 12, pBj136-28 (628); lane 13, pBj136-35 (635); lane 14, pBj136-13 (613); lane 15, vector (pSa13)-transformed control; lane 16, pBj134-27 (427); lane 17, pBj134-34 (434); lane 18, pBj134-38 (438); lane 19, pBj136-22 (622); lane 20, pBj134-39 (439); lane 21, pBj134-44 (444); lane 22, pBj134-45 (445). Two lines with single-copy inserts of OE-wtBjHMGS1 “430” and “445” (lanes 1 and 22) and OE-S359A “622” and “625” (lanes 5 and 19) were used in further tests (underlined). Plasmid pBj134 is a pSa13 derivative containing the 35S::wild-type BjHMGS1 (wtBjHMGS1) fusion (Wang et al., Plant Biotechnol. J. 10:31-42, 2012), and plasmid pBj136 is a pSa13 derivative containing the 35S::mutant BjHMGS1 (S359A) fusion (Wang et al., Plant Biotechnol. J. 10:31-42, 2012).

FIG. 13 shows comparison in growth between tomato HMGS-OE plants and vector-transformed control. Panel (a) shows representative greenhouse-grown plants photographed 35-day after germination (Bar=3 cm). Panel (b) shows the statistical analysis on height of 35-day-old transgenic plants. Panel (c) shows representative greenhouse-grown plants photographed 63-day after germination (Bar=6 cm). Panel (d) shows the statistical analysis on height of 63-day-old transgenic plants. Values are mean±SD (n=30); Bars are SD; **, P<0.01 by the Student's t-test. The vector-transformed control is labelled “pSa13”, two independent lines of OE-wtBjHMGS1 plants are labelled “430” and “445” and two independent lines of OE-S359A plants are labelled “622” and “625”.

### DETAILED DESCRIPTION OF THE INVENTION

Provided herein is a transgenic plant, seed or progeny thereof genetically engineered to overexpress one or more exogenous 3-hydroxy-3-methylglutaryl-coA synthase 1 (HMGS1) in an amount effective to enhance growth and/or seed yield as compared to a control plant. The transgenic plant belongs to the Solanaceae family, and the one or more exogenous HMGS1 comprises an amino acid sequence at least 77% identical to the Brassica juncea HMGS1 protein as set forth in SEQ ID NO:6. Also provided is a plant product, e.g., a commodity product, derived from the transgenic plant, which product overexpresses the one or more exogenous HMGS1.

In one embodiment, the transgenic plant/seed/progeny/plant product comprises one or more exogenous nucleic acid sequences encoding one or more HMGS1. The one or more HMGS1 comprises an amino acid sequence at least 77% identical to SEQ ID NO:6. For instance, the transgenic plant/seed/progeny comprises an exogenous nucleic acid sequence encoding a HMGS1 that comprises an amino acid sequence as set forth in SEQ ID NO:6 except that the amino acid residue serine at position 359 is changed to amino acid residue alanine (i.e., overexpressing HMGS1 mutant S359A). Another example is that the transgenic plant/seed/progeny/plant product comprises an exogenous nucleic acid sequence, wherein the exogenous nucleic acid sequence encodes a HMGS1 comprising an amino acid sequence as set forth in SEQ ID NO:6 except that the amino acid residue histidine at position 188 is changed to amino acid residue asparagine and the amino acid residue serine at position 359 is changed to amino acid residue alanine (i.e., overexpressing HMGS1 mutant H188N/S359A). Still another example is that the transgenic plant/seed/progeny/plant product comprises an exogenous nucleic acid sequence encoding a HMGS1 that comprises an amino acid sequence as set forth in SEQ ID NO:6 (i.e., overexpressing wild-type HMGS1).

In one embodiment, the transgenic plant is selected from the group consisting of tobacco, potato, tomato, pepper, and eggplant. For example, the transgenic plant is tobacco or tomato.

Also provided herein is a method of enhancing plant growth and/or seed yield. Such method comprises genetically engineering a plant to overexpress one or more exogenous HMGS1 in an amount effective to enhance growth and/or seed yield relative to a control plant. The one or more exogenous HMGS1 comprise an amino acid sequence at least 77% identical to SEQ ID NO:6.

In one embodiment, the method comprises the steps of transforming a plant with a vector comprising one or more exogenous nucleic acid sequences encoding the one or more exogenous HMGS1 operably linked to one or more plant expressible promoter; and expressing the one or more exogenous HMGS1 in the plant in an amount effective to provide enhanced growth and/or seed yield relative to a control plant. For instance, the vector comprises an exogenous nucleic acid sequence encoding a HMGS1 that comprises an amino acid sequence as set forth in SEQ ID NO:6 except that the amino acid residue serine at position 359 is changed to amino acid residue alanine (i.e., overexpressing HMGS1 mutant S359A). Another example is that the vector comprises an exogenous nucleic acid sequences, wherein the exogenous nucleic acid sequence encodes a HMGS1 comprising an amino acid sequence as set forth in SEQ ID NO:6 except that the amino acid residue histidine at position 188 is changed to amino acid residue asparagine and the amino acid residue serine at position 359 is changed to amino acid residue alanine (i.e., overexpressing HMGS1 mutant H188N/S359A). Still another example is that the vector comprises an exogenous nucleic acid sequence encoding a HMGS1 that comprises an amino acid sequence as set forth in SEQ ID NO:6 (i.e., overexpressing wild-type HMGS1).

In another embodiment, plant belongs to the Solanaceae family. For example, the plant is tobacco, potato, tomato, pepper, or eggplant.

In still another embodiment, the one or more plant expressible promoter is selected from the group consisting of a constitutive promoter, a tissue-specific promoter and an inducible promoter.

Further provided herein is a method of screening for a functional variant of Brassica juncea HMGS1 that comprises the amino acid sequence as set forth in SEQ ID NO:6. Such method comprises the steps of obtaining a plant cell genetically modified to express a candidate variant; regenerating a plant from the plant cell; and determining whether the plant exhibits an increase in growth and/or seed yield, thereby determining whether the candidate variant is a functional equivalent of the Brassica juncea HMGS1.

In one embodiment, the plant cell belongs to the Solanaceae family.

As used herein, the term “HMGS1” refers to polynucleotides or polypeptides of Brassica juncea 3-hydroxy-3-methylglutaryl-CoA synthase 1 and functional variants thereof (such as H188N, S359A, H188N/S359A) that can convey improved growth and/or seed yield to the host in which they are expressed.

As used herein, the term “HMGS1-OEs” refers to transgenic Brassica juncea overexpressing the HMGS1 polypeptide.

As used herein, the term “HMGS1-like polypeptide” refers to polypeptides sharing at least 77% sequence identity to HMGS1 that convey improved growth and/or seed yield to the host cell, including variants of HMGS1.

As used herein, the terms “HMGS1-like polypeptide”, “HMGS1 variants” and “HMGS1 homologs” refer to polypeptides that are functional equivalents of HMGS1, which are capable of up-regulating downstream genes in the isoprenoid pathway such as NtHMGR1, NtIPI2, NtSQS, NtSMT1-2 and NtCYP85A1.

As used herein, the term “chemically synthesized” means the component nucleotides of a sequence of DNA are assembled in vitro.

As used herein, the term “construct” refers to a recombinant nucleic acid, generally recombinant DNA, which has been generated for the purpose of expressing specific nucleotide sequence(s), or is to be used in the construction of other recombinant nucleotide sequences.

As used herein, the term “cotyledon” refers to the embryonic first leaves of a seedling.

As used herein, the terms “DNA regulatory sequences”, “control elements”, and “regulatory elements” are used interchangeably and refer to transcriptional and translational control sequences, such as promoters, enhancers, polyadenylation signals, terminators, protein degradation signals, and the like, that provide for and/or regulate expression of a coding sequence and/or production of an encoded polypeptide in a host cell.

As used herein, the term “endogenous nucleic acid” refers to a nucleic acid that is normally found in and/or produced by a given bacterium, organism, or cell in nature. An “endogenous nucleic acid” is also referred to as a “native nucleic acid” or a nucleic acid that is “native” to a given bacterium, organism, or cell.

As used herein, the term “exogenous nucleic acid” refers to a nucleic acid that is not normally or naturally found in and/or produced by a given bacterium, organism, or cell in nature.

As used herein, the term “heterologous nucleic acid” refers to a nucleic acid wherein at least one of the following is true: (a) the nucleic acid is foreign (“exogenous,” i.e., not naturally found in a given host microorganism or host cell); (b) the nucleic acid comprises a nucleotide sequence that is naturally found in (i.e., “endogenous to”) a given host microorganism or host cell but produced in an unnatural amount in the cell (e.g., greater than expected or greater than naturally found); (c) the nucleic acid comprises a nucleotide sequence that differs from the endogenous nucleotide sequence but encodes the same protein (i.e., having the same or substantially the same amino acid sequence) and produced in an unnatural amount in a host cell; (d) the nucleic acid comprises two or more nucleotide sequences that are not found in the same relationship in nature, e.g., the nucleic acid is recombinant. An example of a heterologous nucleic acid is a nucleotide sequence encoding an HMGS1 operably linked to a transcriptional control element (e.g., a promoter) to which an endogenous HMGS1 coding sequence is not normally operably linked. Another example of a heterologous nucleic acid is a high copy number plasmid comprising a nucleotide sequence encoding an HMGS1. Still another example of a heterologous nucleic acid is a nucleic acid encoding an HMGS1, where a host cell that does not normally produce HMGS1 is genetically modified with the nucleic acid encoding HMGS1. In this case, because HMGS1-encoding nucleic acids are not naturally found in the host cell, the nucleic acid is heterologous to the genetically modified host cell.

As used herein, the term “host cell” refers to an in vivo or in vitro eukaryotic cell, a prokaryotic cell, or a cell from a multicellular organism (e.g., a cell line) cultured as a unicellular entity, which eukaryotic or prokaryotic cells can be, or have been, used as recipients for a nucleic acid (e.g., an expression vector that comprises a nucleotide sequence encoding one or more gene products such as HMGSs). It is intended to include the progeny of the original cell which has been genetically modified by the nucleic acid. It is understood that the progeny of a single cell may not necessarily be completely identical in morphology or in genomic or total DNA complement as the original parent, due to natural, accidental, or deliberate mutation.

As used herein, the term “a recombinant host cell” or “a genetically modified host cell” refers to a host cell into which a heterologous nucleic acid has been introduced, e.g., via an expression vector.

As used herein, the term “isolated” is meant to describe a polynucleotide, a polypeptide, or a cell, that is in an environment different from that in which the polynucleotide, the polypeptide, or the cell naturally occurs. An isolated genetically modified host cell may be present in a mixed population of genetically modified host cells.

As used herein, the term “naturally-occurring” or “native”, as applied to a nucleic acid, a cell, or an organism, refers to a nucleic acid, cell, or organism that is found in nature. For example, a polypeptide or polynucleotide sequence that can be isolated from a source in nature and has not been intentionally modified by human in the laboratory is naturally occurring; or, “wild-type” plants are naturally occurring.

As used herein, the term “modified plant or plant parts” refers to a plant or plant part, whether it is attached or detached from the whole plant. It also includes progeny of the modified plant or plant parts that are produced through sexual or asexual reproduction.

As used herein, the term “operably linked” refers to a juxtaposition wherein the components are in a relationship permitting them to function in their intended manner. For example, a promoter is operably linked to a coding sequence if the promoter affects its transcription or expression.

As used herein, the term “operon” or “single transcription unit” refers to two or more contiguous coding regions that are coordinately regulated by the same one or more controlling elements (e.g., a promoter).

As used herein, the term “gene product” refers to RNA encoded by DNA (or vice versa) or protein that is encoded by an RNA or DNA, where a gene will typically comprise one or more nucleotide sequences that encode a protein, and may also include introns and other non-coding nucleotide sequences.

As used herein, the term “peptide”, “polypeptide” or “protein” refers to a polymeric form of amino acids of any length, which can include coded and non-coded amino acids, chemically or biochemically modified or derivatized amino acids, and/or polypeptides having modified peptide backbones.

As used herein, the term “percent of sequence identity” of a polypeptide or polynucleotide to another polynucleotide or polypeptide, means that, when aligned, that percentage of bases or amino acids are the same, and in the same relative position, when comparing the two sequences.

As used herein, the term “plant cell culture” refers to cultures of plant units such as protoplasts, cell culture cells, cells in plant tissues, pollen, pollen tubes, ovules, embryo sacs, zygotes and embryos at various stages of development.

As used herein, the term “plant material” refers to leaves, stems, roots, flowers or flower parts, fruits, pollen, egg cells, zygotes, seeds, cuttings, cell or tissue cultures, or any other part or product of a plant.

As used herein, the term “a plant product”, other than a seed or a fruit or vegetable, is intended as a commodity or other products which move through commerce and are derived from a transgenic plant or transgenic plant part, in which the commodity or other products can be tracked through commerce by detecting nucleotide segments, RNA or proteins that encode or comprise distinguishing portions of the proteins of the present disclosure and are produced in or maintained in the plant or plant tissue or part from which the commodity or other product has been obtained. Such commodity or other products of commerce include, but are not limited to, plant parts, biomass, oil, meal, sugar, animal feed, flour, flakes, bran, lint, and processed seed. Plant parts include but are not limited to a plant seed, boll, leaf, flower, stem, pollen, or root. In certain embodiments, the plant part is a non-regenerable portion of said seed, boll, leaf, flower, stem, pollen, or root.

As used herein, the term “plant tissue” refers to a group of plant cells organized into a structural and functional unit. It is intended to include any tissue of a plant, whether in a plant or in culture. It includes, but not limited to, whole plants, plant organs, plant seeds, tissue culture and any groups of plant cells organized into structural and/or functional units. The use of this term in conjunction with, or in the absence of, any specific type of plant tissue as listed above or otherwise embraced by this definition is not intended to be exclusive of any other type of plant tissue.

As used herein, the term “polynucleotide” or “nucleic acid” refers to a polymeric form of nucleotides of any length, either ribonucleotides or deoxyribonucleotides. It includes, but not limited to, single-, double-, or multi-stranded DNA or RNA, genomic DNA, cDNA, DNA-RNA hybrids, or a polymer comprising purine and pyrimidine bases or other natural, chemically or biochemically modified, non-natural or derivatized nucleotide bases.

As used herein, the term “progeny” includes the immediate and all subsequent generations of offspring traceable to a parent.

As used herein, the term “recombinant” means that a particular nucleic acid (DNA or RNA) is the product of various combinations of cloning, restriction, and/or ligation steps resulting in a construct having a structural coding or non-coding sequence distinguishable from endogenous nucleic acids found in natural systems. Generally, DNA sequences encoding the structural coding sequence can be assembled from cDNA fragments and short oligonucleotide linkers, or from a series of synthetic oligonucleotides, to provide a synthetic nucleic acid which is capable of being expressed from a recombinant transcriptional unit contained in a cell or in a cell-free transcription and translation system. Such sequences can be provided in the form of an open reading frame uninterrupted by internal non-translated sequences, or introns, which are typically present in eukaryotic genes. Genomic DNA comprising the relevant sequences can also be used in the formation of a recombinant gene or transcriptional unit. Sequences of non-translated DNA may be present at 5′ or 3′ from the open reading frame, where such sequences do not interfere with manipulation or expression of the coding regions, and may indeed act to modulate production of a desired product by various mechanisms (see “DNA regulatory sequences”, below). Thus, as used herein, the term “recombinant” polynucleotide or nucleic acid refers to one which is not naturally occurring (e.g., made by artificial combination of two otherwise separated segments of sequence through human intervention). This artificial combination is often accomplished by either chemical synthesis means, or by the artificial manipulation of isolated segments of nucleic acids, e.g., by genetic engineering techniques. Such is usually done to replace a codon with a redundant codon encoding the same or a conservative amino acid, while typically introducing or removing a sequence recognition site. Alternatively, it is performed to join together nucleic acid segments of desired functions to generate a desired combination of functions.

As used herein, the term “transformation” or “transformed” is used interchangeably herein with the term “genetic modification” or “genetically modified” and refer to a permanent or transient genetic change induced in a cell following introduction of new nucleic acid (i.e., DNA exogenous to the cell). Genetic change (“modification”) can be accomplished either by incorporation of the new DNA into the genome of the host cell, or by transient or stable maintenance of the new DNA as an episomal element. Where the cell is a eukaryotic cell, a permanent genetic change is generally achieved by introduction of the DNA into the genome of the cell or into a plastome of the cell. In prokaryotic cells, permanent changes can be introduced into the chromosome or via extrachromosomal elements such as plasmids, plastids, and expression vectors, which may contain one or more selectable markers to aid in their maintenance in the recombinant host cell.

As used herein, the term “transformation vectors” or “expression cassettes” refers to a nucleic acid sequence encoding an HMGS1 polypeptide or a functional variant of HMGS1 thereof. The vector or expression cassette can optionally comprise a plant expressible promoter, operably linked to the coding sequence, and a terminator, and/or other regulatory elements. In other embodiments, the vector can be designed to introduce the heterologous polypeptide so that it will be expressed under the control of a plant's own endogenous promoter. The plant transformation vectors preferably include a transcriptional initiation, control region(s) and/or termination region. Transcriptional control regions include those that provide for over-expression of the protein of interest in the genetically modified host cell, and/or those that provide for inducible expression, such that when an inducing agent is added to the culture medium, transcription of the coding region of the protein of interest is induced or increased to a higher level than prior to induction.

As used herein, the term “synthetic nucleic acids” can be assembled from oligonucleotide building blocks that are chemically synthesized using procedures known to those skilled in the art. These building blocks are ligated and annealed to form gene segments which are then enzymatically assembled to construct the entire gene.

As used herein, the term “variant” refers either to a naturally occurring genetic mutant of HMGS1 or a recombinantly prepared variation of HMGS1, each of which contain one or more mutations in its DNA. The term “variant” may also refer to either a naturally occurring variation of a given peptide or a recombinantly prepared variation of a given peptide or protein in which one or more amino acid residues have been modified by amino acid substitution, addition, or deletion.

As used herein, the term “a control plant” refers to vector (pSa13)-transformed plant, wherein the HMGS1 polypeptide is not overexpressed.

Vectors/Expression Cassettes for Enhancing Plant Growth and/or Seed Yield

The plant transformation vectors/expression cassettes used herein included one or more nucleic acid sequences encoding one or more HMGS1 polypeptides or a functional variant thereof, operably linked to a plant expressible promoter, a terminator, and/or other regulatory elements. In one embodiment, the expression cassette comprises operatively linked in the 5′ to 3′ direction, a promoter; one or more nucleic acid sequence encoding an HMGS1 or a functional variant or fragment of HMGS1; and a 3′ polyadenylation signal. In another embodiment, the expression cassette comprises more than one HMGS1 or a functional variant of HMGS1 thereof expressed as an operon, wherein the coding nucleotide sequences can be operably linked to the same promoter. Alternatively, the coding nucleotide sequences may be under the control of different promoters.

Several plant transformation vector options are available, including those described in “Gene Transfer to Plants” (Potrykus, et al., eds. Springer-Verlag Berlin Heidelberg New York (1995)); “Transgenic Plants: A Production System for Industrial and Pharmaceutical Proteins” (Owen, et al., eds. John Wiley & Sons Ltd. England (1996)); and “Methods in Plant Molecular Biology: A Laboratory Course Manual” (Maliga, et al. eds. Cold Spring Laboratory Press, New York (1995)). Plant transformation vectors generally include one or more coding sequences of interest under the transcriptional control of 5′ and 3′ regulatory sequences, including a promoter, a transcription termination and/or polyadenylation signal, and a selectable or screenable marker gene. For the expression of two or more polypeptides from a single transcript, additional RNA processing signals and ribozyme sequences can be engineered into the construct (see, e.g., U.S. Pat. No. 5,519,164). This approach locates multiple transgenes in a single locus, which is advantageous in subsequent plant breeding efforts.

For direct expression of transgenes from the plastid genome, a vector to transform the plant plastid chromosome by homologous recombination is used in which case it is possible to take advantage of the prokaryotic nature of the plastid genome and insert a number of transgenes as an operon (see, e.g., U.S. Pat. No. 5,545,818; WO 2010/061186). WO 2010/061186 describes an alternative method for introducing genes into the plastid chromosome using an adapted endogenous cellular process for the transfer of RNAs from the cytoplasm to the plastid where they are incorporated by homologous recombination. This plastid transformation procedure is also suitable for practicing the disclosed compositions and methods.

### A. HMGS1

Genes or cDNAs encoding HMGS1 useful in the vectors described herein include naturally occurring HMGS1 (GenBank/EMBL data library under accession numbers AF148847). Other genes useful for conferring enhanced growth and/or seed yield to plants include variants of HMGS1. In some embodiments, the variant is a synthetic nucleic acid, which includes less than 25, less than 20, less than 15, less than 10, less than 5, less than 4, less than 3, or less than 2 amino acid substitutions, rearrangements, insertions, and/or deletions relative to Brassica juncea HMGS1. In this regard, the term “variant” is intended to encompass fragments, derivatives, and homologs of Brassica juncea HMGS1 that exhibits the same function as HMGS1. A HMGS1 homolog is preferably a HMGS1-like sequence with at least 77% DNA homology to HMGS1 and is capable of up-regulating downstream genes in the isoprenoid pathway such as NtHMGR1, NtIPI2, NtSQS, NtSMT1-2 and NtCYP85A1. More preferably, the variants comprise peptide sequences having at least 90% amino acid sequence identity to Brassica juncea HMGS1.

Sequence similarity can be determined using methods known in the art. For example, determine sequence identity, sequences can be aligned using the methods and computer programs, including BLAST (see, e.g., Altschul, et al. J. Mol. Biol. 215: 403-410 (1990)). Another alignment algorithm is FASTA, available in the Genetics Computing Group (GCG) package, from Madison, Wis., USA, a wholly owned subsidiary of Oxford Molecular Group, Inc. Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, Calif., USA. Of particular interest are alignment programs that permit gaps in the sequence, e.g., the Smith-Waterman algorithm (Meth. Mol. Biol., 70: 173-187 (1997)). Also, the GAP program using the Needleman and Wunsch alignment method can be utilized to align sequences (J. Mol. Biol., 48: 443-453 (1970)).

In other embodiments, a variant of HMGS1 is a mutant, isolated from a host cell as described herein. In still other embodiments, a variant HMGS1 is encoded by a nucleic acid that hybridizes under stringent conditions to a nucleic acid encoding Brassica juncea HMGS1 or another HMGS1 known in the art.

The coding sequence of the selected gene may be genetically engineered by altering the coding sequence for increased or optimal expression in the crop species of interest. Methods for modifying coding sequences to achieve optimal expression in a particular crop species are well known (see, e.g., Perlak, et al., Proc. Natl. Acad. Sci. USA, 88: 3324 (1991); and Koziel, et al, Biotechnol. 11: 194 (1993)).

### B. Promoters

The selection of promoter used in expression cassettes determines the spatial and temporal expression pattern of the transgene in the transgenic plant. Promoters vary in their strength, i.e., ability to promote transcription. Selected promoters express transgenes in specific cell types (such as leaf epidermal cells, mesophyll cells, root cortex cells) or in specific tissues or organs (such as roots, leaves or flowers) and the selection reflects the desired location of accumulation of the gene product. Alternatively, the selected promoter drives expression of the gene under various inducing conditions.

Various types of plant expressible promoters are suitable herein, such as constitutive promoters, tissue-specific promoters and inducible promoters. Examples of suitable constitutive promoters for nuclear-encoded expression include, but are not limited, the core promoter of the Rsyn7 promoter and other constitutive promoters disclosed in U.S. Pat. No. 6,072,050; the core CaMV 35S promoter, (Odell, et al., Nature 313:810-812 (1985)); rice actin (McElroy, et al., Plant Cell 2:163-171 (1990)); ubiquitin (Christensen, et al., Plant Mol. Biol., 12:619-632 (1989); and Christensen, et al., Plant Mal. Biol. 18:675-689 (1992)); pEMU (Last, et al., Theor. Appl. Genet. 81:581-588 (1991)); MAS (Velten, et al., EMBO J., 3:2723-2730 (1984)); and ALS promoter (U.S. Pat. No. 5,659,026). Still other constitutive promoters were described in U.S. Pat. Nos. 5,608,149; 5,608,144; 5,604,121; 5,569,597; 5,466,785; 5,399,680; 5,268,463; 5,608,142.

Tissue-specific or tissue-preferred promoters can be used to target a gene expression within a particular tissue such as seed, leaf or root tissue. Tissue-preferred promoters are described in Yamamoto, et al., Plant J. 12(2)255-265 (1997); Kawamata, et al., Plant Cell Physiol. 38(7):792-803 (1997); Hansen, et al., Mol. Gen. Genet. 254(3):337-343 (1997); Russell, et al., Transgenic Res. 6(2):157-168 (1997); Rinehart, et al., Plant Physiol. 112(3):1331-1341 (1996); Van Camp, et al., Plant Physiol. 112(2):525-535 (1996); Canevascini, et al., Plant Physiol. 112(2):513-524 (1996); Yamamoto, et al., Plant Cell Physiol. 35(5):773-778 (1994); Lam, Results Probl. Cell Differ. 20:181-196 (1994); Orozco, et al., Plant Mol. Biol. 23(6):1129-1138 (1993); Matsuoka, et al., Proc Natl. Acad. Sci. USA 90(20):9586-9590 (1993); and Guevara-Garcia, et al., Plant J. 4(3):495-505 (1993). Suitable tissue-specific expression patterns include green tissue specific, root specific, stem specific, and flower specific.

Promoters suitable for expression in green tissue include many which regulate genes involved in photosynthesis. Many of green tissue specific promoters have been cloned from both monocotyledons and dicotyledons, e.g., leaf-specific promoters are known in the art (Yamamoto, et al., Plant J. 12(2):255-265 (1997); Kwon, et al., Plant Physiol. 105:357-67 (1994); Yamamoto, et al. Plant Cell Physiol. 35(5):773-778 (1994); Gotor, et al. Plant J. 3:509-18 (1993); Orozco, et al., Plant Mol. Biol. 23(6):1129-1138 (1993); and Matsuoka, et al. Proc. Natl. Acad. Sci. USA 90(20):9586-9590 (1993). Another example is the maize PEPC promoter from the phosphoenol carboxylase gene (Hudspeth & Grula, Plant Mole. Biol. 12: 579-589 (1989)), and promoters include those encoding rbsC (Coruzzi et al., EMBO J., 3:1671-1697 (1984)).

Suitable root-preferred promoters may be selected from the ones known and widely available in the art or isolated de novo from various compatible species (see, e.g., Hire et al. Plant Mol. Biol. 20(2): 207-218 (1992)—soybean root-specific glutamine synthetase gene; Keller and Baumgartner, Plant Cell, 3(10):1051-1061 (1991)—root-specific control element in the GRP 1.8 gene of French bean; Sanger et al., Plant Mol. Biol. 14(3):433-443 (1990)—root-specific promoter of the mannopine synthase (MAS) gene of Agrobacterium tumefaciens; and Miao et al., Plant Cell, 3(1):1 1-22 (1991)—full-length cDNA clone encoding cytosolic glutamine synthetase (GS), which is expressed in roots and root nodules of soybean; also U.S. Pat. Nos. 5,837,876; 5,750,386; 5,633,363; 5,459,252; 5,401,836; 5,110,732; 5,023,179 and 7,285,656). Particularly, a suitable promoter for root-specific expression is from the SAHH or SHMT promoter as described by Sivanandan et al., Biochimica et Biophysica Acta, 1731:202-208, 2005. Also, the Cauliflower Mosaic Virus (CaMV) 35S promoter has been reported to have root-specific and leaf-specific modules in its promoter region (Benfey et al., EMBO 1, 8:2195-2202, 1989).

A suitable stem-specific promoter is that described in U.S. Pat. No. 5,625,136, which drives expression of the maize trpA gene. Plastid specific promoters include the PrbcL promoter (Allison, et al., EMBO J. 15:2802-2809 (1996); Shiina, et al., Plant Cell, 10: 1713-1722 (1998)); the PpsbA promoter (Agrawal, et al., Nucleic Acids Research, 29: 1835-1843 (2001)); the Prrn 16 promoter (Svab & Maliga, Proc. Natl. Acad. Sci. USA 90: 913-917 (1993), Allison, et al., EMBO J. 15: 2802-2809 (1996)); the PaccD promoter (WO 97/06250; Hajdukiewicz, et al., EMBO J. 16: 4041-4048 (1997)).

Inducible promoters such as chemical-regulated promoters can be used to modulate the expression of a gene in a plant through the application of an exogenous chemical regulator. Depending upon the objective, the promoter may be a chemical-inducible promoter, where application of the chemical induces gene expression, or a chemical-repressible promoter, where application of the chemical represses gene expression. Inducible promoters are well known and widely available to those of ordinary skill in the art, which were used successfully in plants (Padidam, Curr. Opin. Plant Biol. 6:169 (2003); Wang, et al. Trans. Res.: 12, 529 (2003); Gatz and Lenk, Trends Plant Sci. 3:352 (1998)). These inducible systems may be activated by chemicals such as tetracycline, pristamycin, pathogen, light, glucocorticoid, estrogen, copper, herbicide safener, ethanol, IPTG (iso-propyl β-D-1-thiogalactopyranoside), and pathogens. Suitable chemical-inducible promoters include, but are not limited to, the maize Int-2 promoter, which is activated by benzenesulfonamide herbicide safeners; the maize GST promoter, which is activated by hydrophobic electrophilic compounds that are used as pre-emergent herbicides; and the tobacco PR-la promoter, which is activated by salicylic acid. Other chemical-regulated promoters of interest include steroid-responsive promoters (see, e.g., the glucocorticoid-inducible promoter in Schena, et al. Proc. Natl. Acad. Sci. USA, 88:10421-10425 (1991); and McNellis, et al. Plant J., 14(2):247-257(1998)) and tetracycline-inducible and tetracycline-repressible promoters (see, e.g., Gatz, et al., Mol. Gen. Genet. 227:229-237 (1991), and U.S. Pat. Nos. 5,814,618 and 5,789,156), herein incorporated by reference in their entirety.

Another suitable category of inducible promoters is wound inducible promoters. Numerous promoters have been described which are expressed at wound sites, including those described by Stanford, et al., Mod. Gen. Genet. 215:200-208 (1989); Xu, et al., Plant Molec. Biol., 22: 573-588 (1993); Logemann, et al., Plant Cell, 1: 151-158 (1989); Rohrmeier & Lehle, Plant Molec. Biol., 22: 783-792 (1993); Firek, et al., Plant Molec. Biol., 22: 129-142 (1993), and Warner, et al., Plant J., 3: 191-201 (1993).

### C. Transcriptional Terminators

A variety of transcriptional terminators are available for use in expression cassettes, which are responsible for the termination of transcription beyond the transgene and its correct polyadenylation. Accordingly, at the extreme 3′ end of the transcript of the transgene, a polyadenylation signal can be engineered. A polyadenylation signal refers to any sequence that can result in polyadenylation of the mRNA in the nucleus prior to export of the mRNA to the cytosol, such as the 3′ region of nopaline synthase (Bevan, et al. Nucleic Acids Res., 11:369-385 (1983). Other transcriptional terminators are those known to function in plants and include, but are not limited to, the CaMV 35S terminator, the tm1 terminator, the nopaline synthase terminator and the pea rbcS E9 terminator, which are used in both monocotyledonous and dicotyledonous plants.

### D. Other Components of Vectors/Expression Cassettes

Numerous sequences have been found to enhance gene expression from within the transcriptional unit and can be used in conjunction with the genes of interest to increase the gene expression in transgenic plants. For example, various intron sequences such as introns of the maize Adh1 gene have been shown to enhance expression, particularly in monocotyledonous cells. In addition, a number of non-translated leader sequences derived from viruses are also known to enhance expression and are particularly effective in dicotyledonous cells.

The disclosed vectors may further include, within the region that encodes the protein of interest, one or more nucleotide sequences encoding a targeting sequence. A “targeting sequence” is a nucleotide sequence that encodes an amino acid sequence or motif that directs the encoded protein of interest to a particular cellular compartment, resulting in localization or compartmentalization of the protein. Presence of a targeting amino acid sequence in a protein typically results in translocation of all or part of the targeted protein across an organelle membrane and into the organelle interior. Alternatively, the targeting peptide may direct the targeted protein to remain embedded in the organelle membrane. The targeting sequence or region of a targeted protein may contain a string of contiguous amino acids or a group of noncontiguous amino acids. The targeting sequence can be selected to direct the targeted protein to a plant organelle such as a nucleus, a microbody (e.g., a peroxisome, or a specialized version thereof such as a glyoxysome), an endoplasmic reticulum, an endosome, a vacuole, a plasma membrane, a cell wall, mitochondria, a chloroplast or a plastid.

A chloroplast targeting sequence is any peptide sequence that can target a protein to the chloroplasts or plastids, such as the transit peptide of the small subunit of the alfalfa ribulose-biphosphate carboxylase (Khoudi, et al., Gene, 197:343-351 (1997)).

A peroxisomal targeting sequence refers to any peptide sequence, either N-terminal, internal, or C-terminal, that can target a protein to the peroxisomes, such as the plant C-terminal targeting tripeptide SKL (Banjoko & Trelease, Plant Physiol., 107:1201-1208 (1995); Wallace, et al., “Plant Organellular Targeting Sequences,” in Plant Molecular Biology, Ed. R. Croy, BIOS Scientific Publishers Limited (1993) pp. 287-288, and peroxisomal targeting in plant is described in Volokita, The Plant J., 361-366 (1991)).

Plastid targeting sequences are known in the art, including the chloroplast small subunit of ribulose-1,5-bisphosphate carboxylase (Rubisco) (de Castro Silva Filho et al. Plant Mol. Biol. 30:769-780 (1996); Schnell et al. J. Biol. Chem. 266(5):3335-3342 (1991)); 5-(enolpyruvyl)shikimate-3-phosphate synthase (EPSPS) (Archer, et al., J. Bioenerg. Biomemb., 22(6):789-810 (1990)); tryptophan synthase (Zhao et al., J. Biol. Chem., 270(11):6081-6087 (1995)); plastocyanin (Lawrence, et al., J. Biol. Chem., 272(33):20357-20363 (1997)); chorismate synthase (Schmidt, et al., J. Biol. Chem., 268(36):27447-27457 (1993)); and the light harvesting chlorophyll a/b binding protein (LHBP) (Lamppa, et al., J. Biol. Chem., 263:14996-14999 (1988)). See also Von Heijne, et al., Plant Mol. Biol. Rep., 9:104-126 (1991); Clark, et al., J. Biol. Chem. 264:17544-17550 (1989); Della-Cioppa et al. Plant Physiol. 84:965-968 (1987); Romer et al., Biochem. Biophys. Res. Commun. 196:1414-1421 (1993); and Shah et al. Science, 233:478-481 (1986). Alternative plastid targeting signals have also been described in the following: US 2008/0263728; Miras, et al., J Biol Chem, 277(49) (2002): 47770-8 (2002); Miras, et al., J Biol Chem, 282: 29482-29492 (2007).

The expression cassettes described herein may further encode a selectable marker to enable selection of transformation events. There are many methods that have been described for the selection of transformed plants (see Mild, et al., Journal of Biotechnology, 107:193-232 (2004)) and references incorporated within). Selectable marker genes that have been used extensively in plants include, but are not limited to, the neomycin phosphotransferase gene nptII (U.S. Pat. Nos. 5,034,322 and 5,530,196), hygromycin resistance gene (U.S. Pat. No. 5,668,298), the bar gene encoding resistance to phosphinothricin (U.S. Pat. No. 5,276,268), the expression of aminoglycoside 3′-adenyltransferase (aadA) to confer spectinomycin resistance (U.S. Pat. No. 5,073,675), the use of inhibition resistant 5-enolpyruvyl-3-phosphoshikimate synthetase (U.S. Pat. No. 4,535,060) and methods for producing glyphosate tolerant plants (U.S. Pat. Nos. 5,463,175 and 7,045,684). Methods of plant selection that do not use antibiotics or herbicides as a selective agent have been previously described and include expression of glucosamine-6-phosphate deaminase to inactive glucosamine in plant selection medium (U.S. Pat. No. 6,444,878), and a positive/negative system that utilizes D-amino acids (Erikson, et al., Nat Biotechnol, 22:455-8 (2004)). European Patent Publication No. EP 0 530 129 describes a positive selection system which enables the transformed plants to outgrow the non-transformed lines by expressing a transgene encoding an enzyme that activates an inactive compound added to the growth media. U.S. Pat. No. 5,767,378 describes the use of mannose or xylose for the positive selection of transgenic plants. Methods for positive selection using sorbitol dehydrogenase to convert sorbitol to fructose for plant growth have also been described (see WO 2010/102293). Screenable marker genes include the β-glucuronidase gene (Jefferson, et al., EMBO J., 6:3901-3907 (1987); U.S. Pat. No. 5,268,463) and native or modified green fluorescent protein gene (Cubitt, et al., Trends Biochem. Sci. 20: 448-455 (1995); Pan, et al., Plant Physiol., 112: 893-900 (1996).

Transformation events can also be selected through visualization of fluorescent proteins such as the fluorescent proteins from the nonbioluminescent Anthozoa species which include DsRed, and a red fluorescent protein from the Discosoma genus of coral (Matz, et al., Nat Biotechnol, 17:969-73 (1999)). An improved version of the DsRed protein has been developed (Bevis and Glick, Nat Biotech, 20:83-87 (2002)) for reducing aggregation of the protein. Visual selection can also be performed with the yellow fluorescent proteins (YFP) including the variant with accelerated maturation of the signal (Nagai, et al., Nat Biotech., 20:87-90 (2002)), the blue fluorescent protein, the cyan fluorescent protein, and the green fluorescent protein (Sheen, et al., Plant J, 8:777-84 (1995); Davis and Vierstra, Plant Molecular Biology, 36:521-528 (1998)). A summary of fluorescent proteins can be found in Tzfira et al. (Tzfira, et al., Plant Molecular Biology, 57:503-516 (2005); and Verkhusha and Lukyanov, Nat Biotech, 22:289-296 (2004), which references are incorporated in entirety). Improved versions of many of the fluorescent proteins have been made for various applications. Use of the improved versions of these proteins or the use of combinations of these proteins for selection of transformants will be obvious to those skilled in the art. It is also practical to simply analyze progeny from transformation events for the presence of the BjHMGS1 thereby avoiding the use of any selectable marker.

For plastid transformation constructs, a preferred selectable marker is the spectinomycin-resistant allele of the plastid 16S ribosomal RNA gene (Staub and Maliga, Plant Cell, 4:39-45 (1992); Svab, et al., Proc. Natl. Acad. Sci. USA, 87: 8526-8530 (1990)). Selectable markers that have since been successfully used in plastid transformation include the bacterial aadA gene that encodes aminoglycoside 3′-adenyltransferase (AadA) conferring spectinomycin and streptomycin resistance (Svab, et al., Proc. Natl. Acad. Sci. USA, 90:913-917 (1993)), nptll that encodes aminoglycoside phosphotransferase for selection on kanamycin (Carrer, et al., Mol. Gen. Genet., 241:49-56 (1993); Lutz, et al., Plant J., 37: 906-913 (2004); Lutz, et al., Plant Physiol., 145:1201-1210 (2007)), aphA6, another aminoglycoside phosphotransferase (Huang, et al, Mol. Genet. Genomics, 268: 19-27 (2002)), and chloramphenicol acetyltransferase (Li, et al. Plant Mol Biol, 76(5-6):443-451 (2010)). Another selection scheme has been reported that uses a chimeric betaine aldehyde dehydrogenase gene (BADH) capable of converting toxic betaine aldehyde to nontoxic glycine betaine (Daniell, et al., Curr. Genet, 39: 109-116 (2001)).

### Transgenic Plants/Plant Materials

A wide variety of plants and plant cells can be engineered to express an HMGS1 polypeptide or a functional fragment or variant of HMGS1. Plant materials such as leaves, stems, roots, flowers or flower parts, fruits, pollen, egg cells, zygotes, seeds, cuttings, cell or tissue cultures, or any other part or product of a plant can thus be obtained, thus genetically modified and exhibiting improved growth and/or seed yield.

The genetically modified plant or plant material comprises one or more genes encoding an HMGS1 polypeptide or a functional fragment or variant of HMGS1. In some embodiments the genetically modified plant/plant material comprises two nucleotide sequences encoding two or more HMGS1, which may be contained on separate expression vectors under the control of separate promoters, or on single expression vector under the control of a common promoter.

In some embodiments, suitable plants and plant cells for engineering include monocotyledonous and dicotyledonous plants, such as grain crops (e.g., wheat, maize, rice, millet, barley), tobacco, fruit crops (e.g., tomato, strawberry, orange, grapefruit, banana), forage crops (e.g., alfalfa), root vegetable crops (e.g., carrot, potato, sugar beets, yam), leafy vegetable crops (e.g., lettuce, spinach); flowering plants (e.g., petunia, rose, chrysanthemum), conifers and pine trees (e.g., pine fir, spruce); oil crops (e.g., sunflower, rape seed); and plants used for experimental purposes (e.g., Arabidopsis). Other examples include plants that are typically grown in groups of more than 10 in order to harvest the entire plant or a part of the plant, e.g., a fruit, a crop, a tree (e.g., fruit trees, trees grown for wood production, trees grown for decoration, etc.), a flower of any kind (e.g., plants grown for purposes of decoration following their harvest), cactuses. Further examples of suitable plants engineered to express HMGSs include Viridiplantae, Streptophyta, Embryophyta, Tracheophyta, Euphyllophytes, Spermatophyta, Magnoliophyta, Liliopsida, Commelinidae, Poales, Poaceae, Oryza, Oryza sativa, Zea, Zea mays, Hordeum, Hordeum vulgare, Triticum, Triticum aestivum, Eudicotyledons, Core eudicots, Asteridae, Euasterids, Rosidae, Eurosids II, Brassicales, Brassicaceae, Arabidopsis, Magnoliopsida, Solananae, Solanales, Solanaceae, Solanum, and Nicotiana. Additional plants that can be transformed using the vectors described herein include, but are not limited to, species from the genera Anacardium, Arachis, Asparagus, Atropa, Avena, Brassica, Citrus, Citrullus, Capsicum, Carthamus, Cocos, Coffea, Cucumis, Cucurbita, Daucus, Elaeis, Fragaria, Glycine, Gossypium, Helianthus, Heterocallis, Hordeum, Hyoscyamus, Lactuca, Linum, Lolium, Lupinus, Lycopersicon, Malus, Manihot, Majorana, Medicago, Nicotiana, Olea, Oryza, Panieum, Panneserum, Persea, Phaseolus, Pistachia, Pisum, Pyrus, Prunus, Raphanus, Ricinus, Secale, Senecio, Sinapis, Solanum, Sorghum, Theobromus, Trigonella, Titicum, Vicia, Vitis, Vigna, and Zea.

Methods for Producing Growth and/or Seed Yield Enhanced Plant/Plant Cells

Transgenic plants and plant cells/plant materials may be obtained by engineering one or more of the vectors expressing an HMGS1 polypeptide or a functional fragment or variant of HMGS1 as described herein into a variety of plant cell types, including but not limited to, protoplasts, tissue culture cells, tissue and organ explants, pollens, embryos, as well as whole plants. Transformation methods as well as methods for introducing nucleotide sequences into plants may vary depending on the type of plant or plant cell targeted for transformation. Suitable methods include microinjection (Crossway, et al., Biotechniques, 4:320-334 (1986)), electroporation (Riggs, et al., Proc. Natl. Acad. Sci. USA, 83:5602-5606 (1986)), Agrobacterium-mediated transformation (Townsend, et al., U.S. Pat. No. 5,563,055; Horsch, et al., Science, 227: 1227-1231 (1985)), direct gene transfer (Paszkowski, et al. EMBO J., 3:2717-2722 (1984)), and ballistic particle acceleration (see, e.g., Sanford et al., U.S. Pat. No. 4,945,050; Tomes, et al., Plant Cell, Tissue, and Organ Culture: Fundamental Methods, ed. Gamborg and Phillips (Springer-Verlag, Berlin) (1995); and McCabe, et al., Biotechnology 6:923-926 (1988)). Also see Weissinger, et al. Ann. Rev. Genet., 22:421-477 (1988); Sanford, et al., Particulate Science and Technology, 5:27-37 (1987) (onion); Christou, et al., Plant Physiol., 87:671-674 (1988) (soybean); McCabe, et al., BioTechnology, 6:923-926 (1988) (soybean); Finer and McMullen, In Vitro Cell Dev. Biol., 27P:175-182 (1991) (soybean); Singh, et al., Theor. Appl. Genet., 96:319-324 (1998) (soybean); Dafta, et al., Biotechnology, 8:736-740 (1990) (rice); Klein, et al., Proc. Natl. Acad. Sci. USA, 85:4305-4309 (1988) (maize); Klein, et al., Biotechnology, 6:559-563 (1988) (maize); Tomes, U.S. Pat. No. 5,240,855; Buising, et al., U.S. Pat. Nos. 5,322,783 and 5,324,646; Tomes, et al. (1995) in Plant Cell, Tissue, and Organ Culture: Fundamental Methods, ed. Gamborg (Springer-Verlag, Berlin) (maize); Klein, et al., Plant Physiol., 91:440-444 (1988) (maize); Fromm, et al., Biotechnology, 8:833-839 (1990) (maize); Hooykaas-Van Slogteren, et al., Nature, 311:763-764 (1984); Bowen, et al., U.S. Pat. No. 5,736,369 (cereals); Bytebier, et al., Proc. Natl. Acad. Sci. USA, 84:5345-5349 (1987) (Liliaceae); De Wet, et al. in The Experimental Manipulation of Ovule Tissues, ed. Chapman et al. (Longman, N.Y.), pp. 197-209 (1985) (pollen); Kaeppler et al. Plant Cell Reports 9:415-418 (1990) and Kaeppler, et al., Theor. Appl. Genet., 84:560-566 (1992) (whisker-mediated transformation); D'Halluin, et al., Plant Cell, 4:1495-1505 (1992) (electroporation); Li, et al., Plant Cell Reports, 12:250-255 (1993); Christou and Ford, Annals of Botany, 75:407-413 (1995) (rice); Osjoda, et al., Nature Biotechnology, 14:745-750 (1996) (maize via Agrobacterium tumefaciens); all of which are herein incorporated by reference in their entirety.

Methods for protoplast transformation and/or gene gun for Agrisoma technology are described in WO 2010/037209. Methods for transforming plant protoplasts are available including transformation using polyethylene glycol (PEG), electroporation, and calcium phosphate precipitation (see e.g., Potrykus, et al., Mol. Gen. Genet., 199:183-188 (1985); Potrykus, et al., Plant Molecular Biology Reporter, 3:117-128 (1985). Methods for plant regeneration from protoplasts have also been described (Evans et al., in Handbook of Plant Cell Culture, Vol 1, (Macmillan Publishing Co., New York, 1983); Vasil, IK in Cell Culture and Somatic Cell Genetics (Academic, Orlando, 1984)).

Methods for transformation of plastids such as chloroplasts are known in the art. See, for example, Svab, et al., Proc. Natl. Acad. Sci. USA, 87:8526-8530 (1990); Svab and Maliga, Proc. Natl. Acad. Sci. USA, 90:913-917 (1993); Svab and Maliga, EMBO J. 12:601-606 (1993) and Staub and Maliga, Plant J. 6:547-553 (1994); Kuehnle, US Publication No. 2009/7618819. The method relies on particle gun delivery of DNA containing a selectable marker and targeting of the DNA to the plastid genome through homologous recombination. Additionally, plastid transformation may be accomplished by transactivation of a silent plastid-borne transgene by tissue-preferred expression of a nuclear-encoded and plastid-directed RNA polymerase (McBride, et al., Proc. Natl. Acad. Sci. USA, 91:7301-7305 (1994)) or by use of an integrase, such as the phiC31 phage site-specific integrase, to target the gene insertion to a previously inserted phage attachment site (Lutz, et al., Plant J, 37:906-13 (2004)). Plastid transformation vectors can be designed such that the transgenes are expressed from a promoter sequence that has been inserted with the transgene during the plastid transformation process, or alternatively, from an endogenous plastidial promoter such that an extension of an existing plastidial operon is achieved (Herz, et al., Transgenic Research, 14:969-982 (2005)). An alternative method for plastid transformation as described in WO 2010/061186, wherein RNA produced in the nucleus of a plant cell can be targeted to the plastid genome, can also be used. Inducible gene expression from the plastid genome using a synthetic riboswitch has also been reported (Verhounig, et al., Proc Natl Acad Sci USA, 107: 6204-6209 (2010)). Methods for designing plastid transformation vectors are described by Lutz, et al., Plant Physiol, 145:1201-10 (2007).

Recombinase technologies which are useful for producing the disclosed transgenic plants include the cre-lox, FLP/FRT and Gin systems. Methods by which these technologies can be used for the purpose described herein are described e.g., in U.S. Pat. No. 5,527,695; Dale And Ow, Proc. Natl. Acad. Sci. USA, 88:10558-10562 (1991); Medberry, et al., Nucleic Acids Res. 23:485-490 (1995).

The engineered plant/plant material is selected or screened for transformants following the approaches and methods described below or screening methods known in the art. Following transformation by any one of the methods described above, procedures that can be used to obtain a transformed plant expressing the transgenes include, but are not limited to: selecting the plant cells that have been transformed on a selective medium; regenerating the plant cells that have been transformed to produce differentiated plants; selecting transformed plants expressing the transgene producing the desired level of desired polypeptide(s) in the desired tissue and cellular location.

A transformed plant cell, callus, tissue, or plant may be identified and isolated by selecting or screening the engineered plant material for traits encoded by the selection marker genes present on the introduced expression cassette. For instance, selection may be performed by growing the engineered plant material on media containing inhibitory amount of the antibiotic or herbicide to which the transforming gene construct confers resistance. Particularly, the selectable marker gene nptII, which specifies kanamycin-resistance, can be used in nuclear transformation. As another example, transformed plants and plant materials may be identified by screening for the activities of any visible marker genes (e.g., the β-glucuronidase, luciferase, B or Cl genes) that may be present on the vectors described herein. Such selection and screening methodologies are well known to those skilled in the art. Alternatively or in addition, the transformed plant cell, callus, tissue, or plant screening may be screened for improved growth and/or seed yield as taught herein.

Physical and biochemical methods may also be used to identify plant or plant cell transformants containing the gene constructs/vectors described herein. These methods include, but are not limited to: 1) Southern analysis or PCR amplification for detecting and determining the structure of the recombinant DNA insert; 2) Northern blot, Si RNase protection, primer-extension or reverse transcriptase-PCR amplification for detecting and examining RNA transcripts of the gene constructs; 3) enzymatic assays for detecting enzyme activity, where such gene products are encoded by the gene construct; 4) protein gel electrophoresis (PAGE), Western blot techniques, immunoprecipitation, or enzyme-linked immunoassays, where the gene construct products are proteins. Additional techniques, such as in situ hybridization, enzyme staining, and immunostaining, may also be used to detect the presence or expression of the recombinant construct in specific plant organs and tissues. The aforementioned methods/assays are well known to those skilled in the art.

The cells that have been transformed may be grown into plants in accordance with conventional techniques. See, e.g., McCormick, et al., Plant Cell Reports 5:81-84(1986). These plants may be grown, pollinated with either the same transformed variety or different varieties, to result in hybrids having constitutive expression of the desired phenotypic characteristic identified. Two or more generations may be grown to ensure that constitutive expression of the desired phenotypic characteristic is stably maintained and inherited, and then seeds are harvested to ensure constitutive expression of the desired phenotypic characteristic. An isolated transformant may be regenerated into a plant and progeny thereof (including the immediate and subsequent generations) via sexual or asexual reproduction or growth. Alternatively, the engineered plant material may be regenerated into a plant before subjecting the derived plant to selection or screening for the marker gene traits. Procedures for regenerating plants from plant cells, tissues or organs, either before or after selecting or screening for marker gene(s), are well known to those skilled in the art.

In plastid transformation procedures, further rounds of regeneration of plants from explants of a transformed plant or tissue can be performed to increase the number of transgenic plastids such that the transformed plant reaches a state of homoplasmy (all plastids contain uniform plastomes containing transgene insert).

Methods for Identifying Genes which Improve Plant Growth and/or Seed Yield

Methods are provided for identifying variants and homologs of HMGS1 that promote plant growth and/or seed yield. An exemplary screening method involves introducing an exogenous nucleic acid into a host cell, producing a test cell, where the host cell is one that exhibits enhanced growth phenotype and reproduction over the wild type. When an exogenous nucleic acid comprising a nucleotide sequence that encodes an HMGS1 or HMGS1-like polypeptide is introduced into the host cell, growth and reproduction of the test cell is enhanced. Thus, an increase in growth and reproduction indicates that the exogenous nucleic acid encodes an HMGS1 or HMGS1-like polypeptide, wherein the encoded polypeptide is produced at a level and/or has an activity that promotes growth and reproduction. The increase in growth and reproduction is observed to be at least about 10%, at least about 20%, at least about 30%, at least about 40%, at least about 50%, at least about 60%, at least about 70%, at least about 80%, at least about 90%, or more, as compared to a non-genetically-modified host.

To generate a genetically modified host cell exhibiting enhanced growth and/or seed yield, one or more nucleic acids including nucleotide sequences encoding one or more HMGS1 polypeptides that could promote growth and reproduction is introduced stably or transiently into a parent host cell, using established techniques, including, but not limited to, electroporation, calcium phosphate precipitation, DEAE-dextran mediated transfection, liposome-mediated transfection, particle bombardment, Agrobacterium-mediated transformation, and the like. For stable transformation, a nucleic acid will generally further include a selectable marker, e.g., neomycin resistance, ampicillin resistance, tetracycline resistance, chloramphenicol resistance, and kanamycin resistance marker.

The exogenous nucleic acid is inserted into an expression vector. Expression vectors that are suitable for use in prokaryotic and eukaryotic host cells are known in the art, including, but not limited to, chromosomal, episomal and virus-derived systems, e.g., vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses and retroviruses, and vectors derived from combinations thereof, such as those derived from plasmid and bacteriophage genetic elements, such as cosmids and phagemids. The expression systems may contain control regions that regulate as well as engender expression. Generally, any system or vector suitable to maintain, propagate or express polynucleotides to produce a polypeptide in a host may be used.

The appropriate nucleotide sequence may be inserted into an expression system by those well-known and routine techniques, e.g., those set forth in Sambrook et al., Molecular Cloning, A Laboratory Manual (2nd Ed., Cold Spring Harbor Laboratory Press, Cold Spring Harbor, N.Y. (1989)). Where a parent host cell has been genetically modified to produce two or more HMGS1s, nucleotide sequences encoding two or more HMGS1s, in some embodiments, are each contained on separate expression vectors; or in other embodiments, are contained on a single expression vector, operably linked to a common control element (e.g., a promoter).

An exogenous nucleic acid, in some embodiments, is isolated from a cell or an organism in its natural environment. Methods of isolating the exogenous nucleic acid from test cell are well known in the art. Suitable methods include, but are not limited to, alkaline lysis methods known in the art. In other embodiments, the nucleic acid of the cell or organism is mutated before it is isolated from the cell or organism. Still in other embodiments, the exogenous nucleic acid is synthesized in a cell-free system in vitro.

In some embodiments, the screening method includes further characterizing a candidate gene product. In these embodiments, the exogenous nucleic acid comprising nucleotide sequence(s) encoding an HMGS(s) are isolated from a test cell as described above. The isolated nucleic acid may be subjected to nucleotide sequence analysis, and the amino acid sequence of the gene product deduced from the nucleotide sequence may further be analyzed as well. In some embodiments, the amino acid sequence of the gene product is compared with other amino acid sequences in a public database of amino acid sequences, to determine whether any significant amino acid sequence identity to an amino acid sequence of a known protein exists.

After the exogenous gene has been identified as having the ability to improve plant growth and/or seed yield, the newly identified HMGS1 variant/homolog can be used to provide plants/plant cells with enhanced growth and/or seed yield.

### Exogenous Nucleic Acids

Exogenous nucleic acids that are suitable for introducing into a host cell, to produce a test cell, include, but are not limited to, naturally-occurring nucleic acids isolated from a cell. Exogenous nucleic acids to be introduced into a host cell may be identified by hybridization under stringent conditions to a nucleic acid encoding HMGS1. Exogenous sequences which show 77% or more nucleotide sequence homology with HMGS1 can also be introduced into a host cell to form a test cell. An HMGS1-like sequence with at least 77% DNA homology to HMGS1, up-regulate downstream genes in the isoprenoid pathway such as NtHMGR1, NtIPI2, NtSQS, NtSMT1-2 and NtCYP85A1 similar to HMGS1, are identified as HMGS1-like polypeptides, variants or homologs. More preferably, the sequence homology is, 80% or greater, 81% or greater, 82% or greater, 83% or greater, 84% or greater, 85% or greater, 86% or greater, 87% or greater, 88% or greater, 89% or greater, 90% or greater, 91% or greater, 92% or greater, 93% or greater, 94% or greater, 95% or greater, 96% or greater, 97% or greater, 98% or greater, 99% or greater.

Naturally-occurring nucleic acids that have been modified (for example, by mutation) before or subsequent to isolation from a cell; synthetic nucleic acids, e.g., nucleic acids synthesized in a laboratory using standard methods of chemical synthesis of nucleic acids, or generated by recombinant methods; synthetic or naturally-occurring nucleic acids that have been amplified in vitro, either within a cell or in a cell-free system; and the like. Exemplary exogenous nucleic acids include, but are not limited to, genomic DNA; RNA; a complementary DNA (cDNA) copy of mRNA isolated from a cell; recombinant DNA; and DNA synthesized in vitro, e.g., using standard cell-free in vitro methods for DNA synthesis. In some embodiments, exogenous nucleic acids are a cDNA library made from cells, either prokaryotic cells or eukaryotic cells. In some embodiments, exogenous nucleic acids are a genomic DNA library made from cells, either prokaryotic cells or eukaryotic cells.

In some embodiments, for example, where the exogenous nucleic acid is a plurality of exogenous nucleic acids (such as, for example, a cDNA library, a genomic library, or a population of nucleic acids, each encoding an HMGS1 or HMGS1-like polypeptide with a different amino acid sequence, etc.), the exogenous nucleic acids are introduced into a plurality of host cells, forming a plurality of test cells. The test cells are in some embodiments grown in culture under normal conditions such that native cells of the same type would exhibit normal growth and reproduction; those test cells comprising an exogenous nucleic acid that comprises nucleotide sequences encoding an HMGS1/HMGS1-like polypeptide will show enhance in growth and reproduction over test cells that do not comprise an exogenous nucleic acid that comprises nucleotide sequences encoding an HMGS1/HMGS1-like polypeptide.

In other embodiments, the exogenous nucleic acid is a synthetic nucleic acid which comprises a nucleotide sequence encoding a variant HMGS1. For example, an HMGS1 that differs in amino acid sequence by one or more amino acids from a naturally-occurring Brassica juncea HMGS1 or other parent HMGS1. In some embodiments, a variant HMGS1 differs in amino acid sequence by one amino acid, two amino acids, three amino acids, four amino acids, five amino acids, six amino acids, seven amino acids, eight amino acids, nine amino acids, or ten amino acids, or more, compared to the amino acid sequence of a naturally-occurring parent HMGS1. In some embodiments, a variant HMGS differs in amino acid sequence by from about 10 amino acids to about 15 amino acids, from about 15 amino acids to about 20 amino acids, from about 20 amino acids to about 25 amino acids, from about 25 amino acids to about 30 amino acids, from about 30 amino acids to about 35 amino acids, from about 35 amino acids to about 40 amino acids, from about 40 amino acids to about 50 amino acids, or from about 50 amino acids to about 60 amino acids, compared to the amino acid sequence of a naturally-occurring parent HMGS1.

Manual chemical synthesis of DNA may be accomplished using well-established procedures, or automated chemical synthesis can be performed using one of a number of commercially available machines. The nucleotide sequence of the nucleic acids can be modified for optimal expression based on optimization of nucleotide sequence to reflect the codon bias of the host cell. The skilled artisan appreciates the likelihood of successful expression if codon usage is biased towards those codons favored by the host. Determination of preferred codons can be based on a survey of genes derived from the host cell where sequence information is available. Fragments of full-length proteins can be produced by techniques well known in the art, such as by creating synthetic nucleic acids encoding the desired portions; or by use of Bal 31 exonuclease to generate fragments of a longer nucleic acid.

In still other embodiments, a variant HMGS1 is encoded by a nucleic acid that hybridizes under stringent conditions to a nucleic acid encoding a Brassica juncea HMGS1 or another HMGS1 known in the art.

Nucleic acids will in some embodiments be mutated before being introduced into a host cell to form the test cell. In these embodiments, a nucleic acid comprising a nucleotide sequence encoding a naturally-occurring HMGS1 is mutated, using any of a variety of well-established methods, giving rise to a nucleic acid comprising a nucleotide sequence encoding a variant HMGS1. Nucleotide sequences encoding HMGSs are known in the art, and any known HMGS1-encoding nucleotide sequence can be altered to generate a synthetic nucleic acid for use in a subject method.

Methods of mutating a nucleic acid are well known in the art and include well-established chemical mutation methods, radiation-induced mutagenesis, and methods of mutating a nucleic acid during synthesis. Chemical methods of mutating DNA include exposure of DNA to a chemical mutagen, e.g., ethyl methanesulfonate (EMS), methyl methanesulfonate (MMS), N-nitrosourea (ENU), N-methyl-N-nitro-N′-nitrosoguanidine, 4-nitroquinoline N-oxide, diethylsulfate, benzopyrene, cyclophosphamide, bleomycin, triethylmelamine, acrylamide monomer, nitrogen mustard, vincristine, diepoxyalkanes (for example, diepoxybutane), ICR-170, formaldehyde, procarbazine hydrochloride, ethylene oxide, dimethylnitrosamine, 7,12 dimethylbenz(a)anthracene, chlorambucil, hexamethylphosphoramide, bisulfan, and the like. Radiation mutation-inducing agents include ultraviolet radiation, .gamma.-irradiation, X-rays, and fast neutron bombardment. Mutations can also be introduced into a nucleic acid using, e.g., trimethylpsoralen with ultraviolet light. Random or targeted insertion of a mobile DNA element, e.g., a transposable element, is another suitable method for generating mutations. Mutations can be introduced into a nucleic acid during amplification in a cell-free in vitro system, e.g., using a polymerase chain reaction (PCR) technique such as error-prone PCR. Mutations can be introduced into a nucleic acid in vitro using DNA shuffling techniques (e.g., exon shuffling, domain swapping, and the like). Mutations can also be introduced into a nucleic acid as a result of a deficiency in a DNA repair enzyme in a cell, e.g., the presence in a cell of a mutant gene encoding a mutant DNA repair enzyme is expected to generate a high frequency of mutations (i.e., about 1 mutation/100 genes-1 mutation/10,000 genes) in the genome of the cell. Examples of genes encoding DNA repair enzymes include but are not limited to Mut H, Mut S, Mut L, and Mut U, and the homologs thereof in other species (e.g., MSH 1 6, PMS 1 2, MLH 1, GTBP, ERCC-1, and the like). Methods of mutating nucleic acids are well known in the art, and any known method is suitable for use. See, e.g., Stemple, Nature Reviews, 5:1-7 (2004); Chiang, et al. PCR Methods Appl., 2(3):210-217 (2003); Stemmer, Proc. Natl. Acad. Sci. USA, 91:10747-10751 (1994); and U.S. Pat. Nos. 6,033,861, and 6,773,900.

Thus, for example, a nucleic acid comprising a nucleotide sequence encoding a naturally-occurring HMGS1 is exposed to a chemical mutagen, as described above, or subjected to radiation mutation, or subjected to an error-prone PCR, and the mutagenized nucleic acid introduced into a genetically modified host cell(s) as described above. Methods for random mutagenesis using a “mutator” strain of bacteria are also well known in the art and can be used to generate a variant HMGS (see, e.g., Greener, et al., Methods in Molecular Biology, 57:375-385 (1995)). Saturation mutagenesis techniques employing a polymerase chain reaction (PCR) are also well known and can be used (see, e.g., U.S. Pat. No. 6,171,820). Nucleic acids comprising a nucleotide sequence encoding a variant HMGS are identified by the ability to relieve growth inhibition caused by lead.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. It should be appreciated by those of ordinary skill in the art that the techniques disclosed in the examples that follow represent approaches the inventors have found function well in the practice of the invention, and thus can be considered to constitute examples of preferred modes for its practice. However, those of ordinary skill in the art should, in light of the present disclosure, appreciate that many changes can be made in the specific embodiments that are disclosed and still obtain a like or similar result without departing from the spirit and scope of the invention.

Unless indicated otherwise, parts are parts by weight, molecular weight is average molecular weight, temperature is in degrees Celsius, and pressure is at or near atmospheric. Standard abbreviations may be used, e.g., bp: base pair(s); kb: kilobase(s); pl: picoliter(s); s or sec: second(s); min: minute(s); h or hr: hour(s); an: amino acid(s); nt: nucleotide(s); and the like.

### Example 1

### Generation and Molecular Analyses of Transgenic Tobacco HMGS-OEs

Plasmids pBj132 (H188N/S359A), pBj134 (wtBjHMGS1), pBj136 (S359A) and pBj137 (H188) were used to transform in Agrobacterium-mediated leaf disc transformation of Nicotiana tabacum L. cv. Xanthi (Wang et al., Plant Biotechnol. J. 10:31-42, (2012)). Putative tobacco HMGS-OEs were denoted as OE-wtBjHMGS1 or “401”, “402” and “404” (wild-type BjHMGS1), OE-H188N (BjHMGS1 H188N), OE-S359A or “602”, “603” and “606” (BjHMGS1 S359A) and OE-H188N/S359A (BjHMGS1 H188N/S359A). Binary vector pSa13 was used to yield the vector-transformed control tobacco lines. Transgenic tobacco, selected on kanamycin contained Murashige and Skoog medium (Murashige and Skoog, Physiol. Plant 15:473-497, (1962)), were transferred to soil for further growth, analysis and seed collection. The BjHMGS1 full-length cDNA is provided below.

(SEQ ID NO: 1)

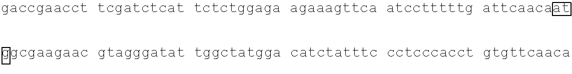

ggaagctttg gaagctcatg atggagcaag caaagggaaa tacaccattg gacttggtca
agattgttta gctttttgca ctgagcttga agatgttatc tcaatgagtt tcaatgcggt
gacatctctt cttgaaaagt acaagattga ccccaagcaa attgggcgtc ttgaagtagg
cagtgagact gtcattgaca agagcaagtc catcaaaacc ttcttgatgc aactctttga
gaaatgtgga aacactgatg tagaaggtgt tgactcgacc aatgcttgtt atggtggaac
tgcggctttg ctaaactgtg tcaattgggt tgagagtaac tcctgggatg gacgctatgg
ccttgtcatt tgtactgaca gtgcggttta tgcagaaggg cccgcaaggc ccactggagg
agcagctgcc attgctatgt tgataggacc agatgctcct attgtttttg agagcaaatt
gagaggaagc cacatggctc atgtctatga cttttacaag ccaaatcttg ctagcgagta
cccggttgtt gatggtaagc tttcacagac atgctacctt atggcacttg actcttgcta
taaacattta tgcaacaagt ttgagaaact tgagggcaaa gagttctcca tcaatgatgc
tgattacttt gtcttccatt ctccatacaa caaacttgtg cagaaaagct ttgctcgtct
cttgtacaac gacttcttga gaaacgcaag ctccattgat gaggctgcca aagaaaagtt
cactccttat tcctccttgt ccctcgatga gagttaccaa agccgtgatc ttgaaaaggt
gtcacaacaa ctagcaaaaa cgtattatga tgctaaagtg caaccaacga ctttagtacc
aaaacaagtc ggtaacatgt acactgcttc tctctacgca gcatttgctt ccctcgtcca
caacaaacac agcgatttgg cgggaaagcg ggtggttatg ttctcatatg gaagtggttc
aaccgcaaca atgttctcat tgcgtctctg cgagaaccag tctcctttca gcttatcaaa
catcgcttct gtaatggatg ttggcggtaa attgaaggct agacatgagt atgcacctga
gaagtttgtg gagacaatga agctaatgga acataggtac ggagcaaagg agtttgtgac
gagcaaggaa ggcatcttag accttttggc tccggggact tattatctga aagaggttga
ttcattgtac cggagattct atggcaagaa aggagacgat ggatccataa ccaatggaca
ctgattcttg aaagcataaa tcacttgaaa gattgtgtct actcattgct ctttatgttt
actttccaaa attctagacg tttttataag gttcttgtta ctttgattta gcgacagttt
gcatgctctt gtttttattt atttgccatt gattctctga agcttttttt ttttctctta
gtgtgtgttt taaataagcc cgttaatttt aggaattatt aagatgttat aataataaac
ctttaa
(Start codon is boxed, stop codon is underlined)

To preliminarily identify tobacco transgenic plants, the 35S promoter forward primer (5′-CAATCCCACTATCCTTCGCAAGACC-3′) (SEQ ID NO:2) and a BjHMGS1 3′-end cDNA reverse primer ML264 (5′-GGATCCATAACCAATGGACACTGAGGATCC-3′) (SEQ ID NO:3) were used to amplify inserts of transgenes. Mutations (H188N and S359A) on the BjHMGS cDNA were validated by DNA sequence analysis on PCR products amplified from total DNA of transgenic plants using primer ML915 (5′-CATTGCTATGTTGATAGGAC-3′) (SEQ ID NO:4).

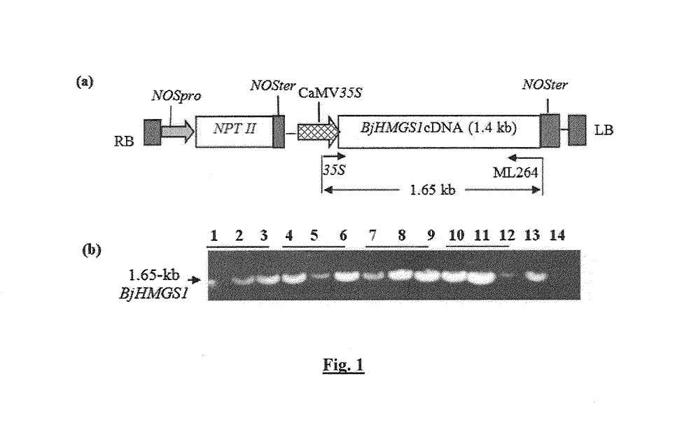

The presence of wild-type and mutant BjHMGS1 in transgenic tobacco was verified by PCR using the 35S promoter forward primer and a BjHMGS1 3′-end cDNA reverse primer ML264 (FIG. 1). DNA sequence analysis was performed on the PCR product amplified from transgenic tobacco to confirm the presence of each mutation (H188N and S359A) in the transgenic mutant lines.

### Example 2

### Western Blot and Northern Blot Analyses of Transgenic Tobacco HMGS-OEs

Total protein was extracted from 3-week-old tobacco leaf. Protein concentration was determined following Bradford using the Bio-Rad Protein Assay Kit I (Bio-Rad). Protein (20 μg) was separated on 12% sodium dodecyl sulfate polyacrylamide gel electrophoresis (SDS-PAGE) and transferred onto Hybond-ECL membrane (Amersham) using a Trans-Blot cell (Bio-Rad) following the instructions of manufacturer. Antibodies against HMGS were used in Western blot analyses according to Wang et al. (Wang et al., Plant Biotechnol. J. 10:31-42, (2012)). A synthetic peptide (DESYQSRDLEKVSQQ) (SEQ ID NO:5) corresponding to BjHMGS1 amino acids 290 to 304 was used for the immunization of rabbits (Wang et al., Plant Biotechnol. J. 10:31-42, (2012)). Western blot assays were carried out according to Xiao et al. (Xiao et al., The Plant Cell 22:1463-1482, (2010)). Cross-reacting bands were detected using the ECL™ Western Blotting Detection Kit (Amersham) following the manufacturer's instructions. The Amino acid sequence of HMGS1 is provided below:

```
(SEQ ID NO: 6)
    1 MAKNVGILAM DIYFPPTCVQ QEALEAHDGA SKGKYTIGLG QDCLAFCTEL EDVISMSFNA
  61 VTSLLEKYKI DPKQIGRLEV GSETVIDKSK SIKTFLMQLF EKCGNTDVEG VDSTNACYGG
121 TAALLNCVNW VESNSWDGRY GLVICTDSAV YAEGPARPTG GAAAIAMLIG PDAPIVFESK

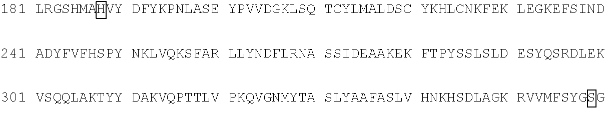

361 STATMFSLRL CENQSPFSLS NIASVMDVGG KLKARHEYAP EKFVETMKLM EHRYGAKEFV
421 TSKEGILDLL APGTYYLKEV DSLYRRFYGK KGDDGSITNG H
(Mutation sites are boxed, H in position 188 is changed to N in the
mutant H188N; S in position 359 is changed to A in the mutant S359A.)
```

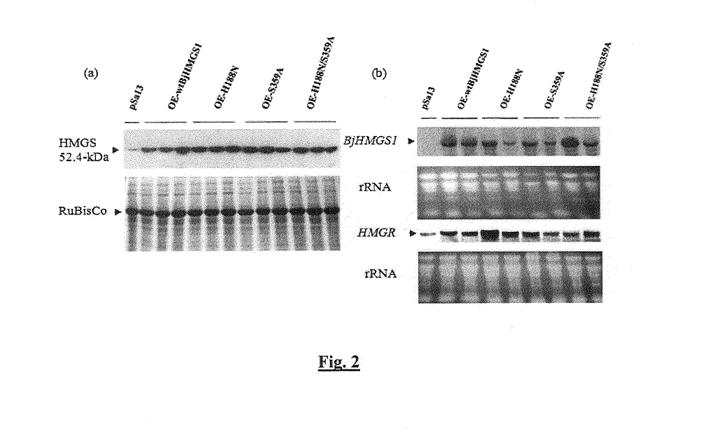

PCR-positive HMGS-OE lines were confirmed by Western blot analysis (FIG. 2, panel (a)). Given that the HMGS-specific peptide used to generate anti-BjHMGS1 antibodies in rabbit shows 100% homology to tobacco HMGS, a faint band was also detected in the vector-transformed control (FIG. 2, panel (a)).

Tobacco total RNA was extracted using a modified cetyltrimethylammonium bromide (CTAB) method. RNA (20 μg per well), separated on 1.3% agarose gels containing 6% formaldehyde, was transferred to Hybond-N membrane (Amersham). Northern blot analysis was performed according to Chen et al. (Chen et al., Plant Physio. 148:304-315, (2008)). DIG-labeled probes were synthesized using the PCR Digoxigenin Probe Synthesis (Roche) with primer pairs ML276, 5′-GGATCCATGGCGAAGAACGTAGGGATATTG-3′ (SEQ ID NO:7) and ML860, 5′-GGAGACTGGTTCTCGCAGAGAC-3′ (SEQ ID NO:8) for BjHMGS1, and ML1046, 5′-CCATAATTACACCAGCAGTGTCC-3′ (SEQ ID NO:9) and ML1047, 5′-CAACTGTGCCAACCTCAATAGAAG-3′ (SEQ ID NO:10) for N. tabacum HMGR1 (GenBank: U60452.1). Hybridization and detection were performed according to Roche. Northern blot analyses revealed that all lines verified by Western blot analysis accumulate BjHMGS1 mRNA (FIG. 2, panel (b)).

### Example 3

### Induced Expression of HMGR mRNA in Tobacco HMGS-OEs

HMGR and HMGS have been reported to be co-regulated in plants and animals (Gil et al., J. Biol. Chem. 261:3710-3716, (1986); Goldstein and Brown, Nature 343:425-430, (1990); Alex et al., Plant J. 22:415-426, (2000)). Since HMGR mRNA has been confirmed to be overexpressed in Arabidopsis HMGS-OEs, its transcription level in tobacco HMGS-OEs was investigated. Results of Northern blot analyses showed that endogenous NtHMGR1 expression was induced in tobacco HMGS-OEs, consistent with results obtained from Arabidopsis HMGS-OEs (FIG. 2, panel (b)).

### Example 4

### Tobacco HMGS-OEs Accumulate Sterols in Both Seedlings and Leaves

Twenty (20) mg of 60-day-old freeze-dried tobacco leaves, 10 mg of 20-day-old freeze-dried tobacco seedlings and 200 mg of freeze-dried fully-opened tobacco flowers were used for sterol profiling. Extraction and quantitative analysis of sterols were then carried out based on art-known protocol (see Babiychuk et al., Proc. Natl. Acad. Sci. USA 105:3163-3168, (2008); Wang et al., Plant Biotechnol. J. 10:31-42, (2012)).

Gas chromatography-mass spectrometry (GC/MS) analyses (GC: Hewlett Packard 6890 with an HP-5MS capillary column: 30 m long, 0.25 mm i.d., film thickness 0.25 μm; MS: Hewlett Packard 5973 mass selective detector, 70 eV) was used to determine the sterol content, with He as the carrier gas (1 ml/min). The column temperature program used included a fast rise from 60° C. to 220° C. (30° C./min) and a slow rise from 220° C. to 300° C. (5° C./min), then kept at 300° C. for 10 min. The inlet temperature was 280° C. Compounds were identified using mass spectral libraries NIST (Agilent, USA). The sterol masses were determined by comparing the peak area of each compound with that of the internal standard. Each sample was injected twice in GC/MS analyses and an average of the sterol mass was taken. The contents of three major sterols (campesterol, stigmasterol and sitosterol) in tobacco HMGS-OEs were subsequently analyzed and compared to those of vector (pSa13)-transformed plants.

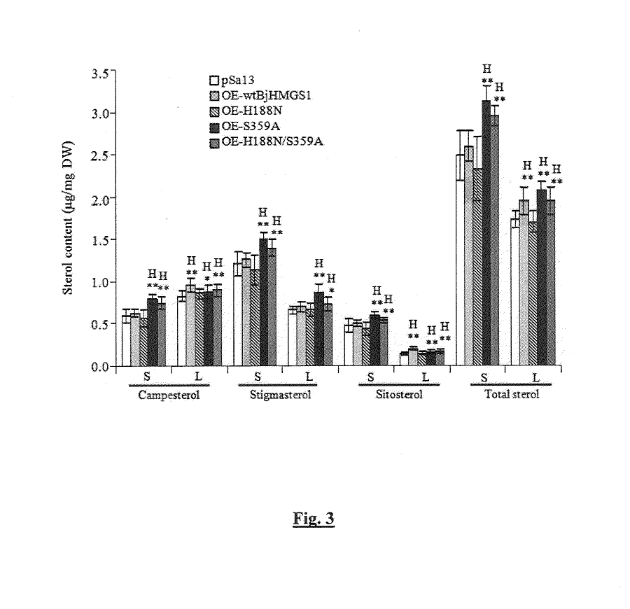

Increase in phytosterol content has been observed in Arabidopsis HMGS-OEs (Wang et al., Plant Biotechnol. J. 10:31-42, (2012)). GC/MS result showed that except for campesterol content, the amounts of sitosterol and stigmasterol in tobacco HMGS-OE seedlings (20-day-old) were higher than in leaves (60-day-old) (Table 1; FIG. 3). Both OE-S359A and OE-H188N/S359A displayed significant higher campesterol, stigmasterol, sitosterol and total sterol contents than the vector-transformed control, as evident from seedlings or leaves (FIG. 3). The increase in sterol content of OE-S359A seedlings (S) and leaves (L) were, campesterol (S: 31.7%; L: 3.5%), stigmasterol (S: 24.0%; L: 31.8%), sitosterol (S: 25.0%; L: 14.3%) and total sterol contents (S: 25.7%; L: 19.0%), followed by OE-H188N/S359A (Table 2). OE-wtBjHMGS1 showed higher campesterol (12.9%), sitosterol (42.9%) and total sterol (12.1%) in leaves, while in seedlings the increases were only 4-5% for each sterol.

TABLE 1
Sterol profiles of tobacco HMGS-OEs (μg/mg dry weight)

| Sterols | pSa13 | OE-wtBjHMGS1 | OE-H188N | OE-S359A | OE-H188N/S359A |
|---|---|---|---|---|---|
| Campesterol (S) | 0.60 ± 0.08 | 0.63 ± 0.05 | 0.57 ± 0.10 | 0.79 ± 0.06^{a} | 0.75 ± 0.07^{a} |
| Campesterol (L) | 0.85 ± 0.06 | 0.96 ± 0.09^{a} | 0.86 ± 0.09 | 0.88 ± 0.07^{b} | 0.90 ± 0.07^{a} |
| Stigmasterol (S) | 1.21 ± 0.15 | 1.26 ± 0.09 | 1.14 ± 0.18 | 1.50 ± 0.09^{a} | 1.40 ± 0.10^{a} |
| Stigmasterol (L) | 0.66 ± 0.05 | 0.70 ± 0.06 | 0.67 ± 0.08 | 0.87 ± 0.10^{a} | 0.73 ± 0.08^{b} |
| Sitosterol (S) | 0.48 ± 0.08 | 0.50 ± 0.04 | 0.44 ± 0.08 | 0.60 ± 0.04^{a} | 0.54 ± 0.03^{a} |
| Sitosterol (L) | 0.14 ± 0.02 | 0.20 ± 0.02^{a} | 0.15 ± 0.02 | 0.16 ± 0.02^{a} | 0.17 ± 0.03^{a} |
| Total sterol (S) | 2.49 ± 0.29 | 2.60 ± 0.18 | 2.33 ± 0.38 | 3.13 ± 0.18^{a} | 2.96 ± 0.12^{a} |
| Total sterol (L) | 1.74 ± 0.10 | 1.95 ± 0.16^{a} | 1.71 ± 0.12 | 2.07 ± 0.11^{a} | 1.95 ± 0.16^{a} |
| C/S (S) | 1.25 | 1.26 | 1.29 | 1.32 | 1.39 |
| C/S (L) | 5.93 | 4.80 | 5.73 | 5.50 | 5.29 |

Two independent lines for each OE genotype were analyzed.
S = 20-day-old seedlings;
L = 60-day-old leaves.
C/S, Campesterol/Sitosterol.
Bold font indicates significantly higher sterol content than the vector (pSa13)-transformed control;
Values are means ± SD,
n = 5;
^{a}P < 0.01;
^{b}P < 0.05 by Student's t-test.

TABLE 2
Increase (%) of sterol composition in tobacco HMGS-OE seedlings
and leaves in comparison to the vector (pSa13)-transformed control
as calculated from Table 1

| | OE- | OE- | | OE-H188N/ |
|---|---|---|---|---|
| Sterols | wtBjHMGS1 | H188N | OE-S359A | S359A |
| Campesterol (S) | 5.0 | −5.0 | 31.7 | 25.0 |
| Campesterol (L) | 12.9 | 1.2 | 3.5 | 5.9 |
| Stigmasterol (S) | 4.1 | −5.8 | 24.0 | 17.3 |
| Stigmasterol (L) | 6.1 | 1.5 | 31.8 | 10.6 |
| Sitosterol (S) | 4.2 | −8.3 | 25.0 | 12.5 |
| Sitosterol (L) | 42.9 | 7.1 | 14.3 | 21.4 |
| Total sterol (S) | 4.4 | −6.4 | 25.7 | 18.9 |
| Total sterol (L) | 12.1 | −1.7 | 19.0 | 12.1 |

Two independent lines for each OE genotype were analyzed.
S = 20-day-old seedlings;
L = 60-day-old leaves.
Values = [(mean_{OEs} − mean_{pSa13})/mean_{pSa13}] * 100.

However, for the OE-H188N line, campesterol, stigmasterol, sitosterol and total sterol contents decreased 5.0%, 5.8%, 8.3% and 6.4%, respectively, in seedlings in comparison to the vector-transformed control (Tables 1 and 2). Sitosterol content displayed a 7.1% increase in leaves, while campesterol and stigmasterol increased only 1.2% and 1.5%, respectively (Tables 1 and 2). This result is consistent with previous results that the enzymatic activity of the single mutant (BjHMGS1 H188N) was 10-fold lower than wild-type BjHMGS1 in E. coli (Nagegowda et al., Biochem. J. 383:517-527, (2004)).

Trace amounts of cholesterol, brassicasterol, isofucosterol, cycloartenol, delta-7-sitosterol, delta-7-avenasterol, 24-methylene cycloartanol and 24-ethylidene lophenol were also detected in all lines tested, but no significant differences between BjHMGS-OEs and vector (pSa13)-transformed controls were observed (data not shown).

### Example 5

### Tobacco HMGS-OEs Accumulate Sterols in Flowers

To investigate how the overexpression of BjHMGS1 led to an increase in tobacco seed yield, the sterol contents in tobacco flowers were analyzed by GC-MS. The results indicate that all the tested tobacco HMGS-OEs displayed significantly higher campesterol, stigmasterol, sitosterol and total sterol contents than wild type. The average increase in sterol content of OE-wtBjHMGS1 lines were, campesterol (20.7%), stigmasterol (11.7%), sitosterol (18.2%) and total sterol contents (18.2%) respectively (Tables 3 and 4). For OE-S359A, campesterol, stigmasterol, sitosterol and total sterol contents increased 13.8%, 18.4%, 18.2% and 18.2% respectively (Tables 3 and 4). However, there was no significant difference in the total sterol of tobacco flowers between OE-wtBjHMGS1 and OE-S359A. The increase in campesterol content of flowers of OE-wtBjHMGS1 was higher than that in OE-S359A, while the increase in stigmasterol content of flowers from tobacco OE-S359A was higher than that of OE-wtBjHMGS1. This result indicated that the sterol accumulation in flowers of tobacco HMGS-OEs culminated in an increase in pod size of tobacco and seed number, suggesting the importance of HMGS in pod and seed production.

TABLE 3
Sterol profiles in flowers of wild type tobacco and HMGS-OEs (μg/mg dry weight)

| Sterols | pSa13 | OE-wtBjHMGS1-1 | OE-wtBjHMGS1-2 | OE-S359A-1 | OE-S359A-2 |
|---|---|---|---|---|---|
| Campesterol | 0.29 ± 0.01 | 0.32 ± 0.02^{b} | 0.38 ± 0.01^{a} | 0.34 ± 0.02^{a} | 0.32 ± 0.01^{b} |
| Stigmasterol | 0.30 ± 0.02 | 0.32 ± 0.02^{b} | 0.35 ± 0.03^{a} | 0.38 ± 0.01^{a} | 0.33 ± 0.02^{b} |
| Sitosterol | 0.33 ± 0.01 | 0.37 ± 0.01^{a} | 0.41 ± 0.02^{a} | 0.41 ± 0.03^{a} | 0.37 ± 0.02^{a} |
| Total sterol | 0.91 ± 0.04 | 1.01 ± 0.05^{b} | 1.14 ± 0.06^{a} | 1.13 ± 0.06^{a} | 1.02 ± 0.05^{b} |

Two independent lines for each OE genotype were analyzed.
pSa13, vector-transformed control.
Values are means ± SD,
n = 3,
^{a}P < 0.01;
^{b}P < 0.05 by the Student's t-test.
Bold font indicates value that is significantly higher than the vector (pSa13)-transformed control.

TABLE 4
Increase (%) of sterol composition in tobacco HMGS-OE flowers
in comparison to the vector (pSa13)-transformed control as
calculated from Table 3

| | OE- | OE- | OE- | OE- |
|---|---|---|---|---|
| Sterols | wtBjHMGS1-1 | wtBjHMGS1-2 | S359A-1 | S359A-2 |
| Campesterol | 10.3 | 31.0 | 17.2 | 10.3 |
| (F) | | | | |
| Stigmasterol | 6.7 | 16.7 | 26.7 | 10 |
| (F) | | | | |
| Sitosterol (F) | 12.1 | 24.2 | 24.2 | 12.1 |
| Total sterol (F) | 11.0 | 25.3 | 24.2 | 12.1 |

Two independent lines for each OE genotype were analyzed.
F = fully-opened flowers.
Values = [(mean_{OEs} − mean_{pSa13})/mean_{pSa13}] * 100

### Example 6

### Tobacco HMGS-OE Plants Show Enhanced Growth

Four-day-old seedlings were transferred onto fresh MS plates for a further 10-day growth, with the plates placed vertically. The fresh weight of these 14-day-old seedlings was then measured. Five (5) fresh seedlings were grouped for weight measurements and a total of 30 groups were analyzed per individual line. The dry weight of 14-day-old lyophilized tobacco seedlings was measured using the same method.

Seven-day-old tobacco seedlings of the same size were chosen and transferred from MS medium to soil for further growth rate measurements. Subsequently, the root length, dry weight (freeze-dried) of 14-day-old tobacco seedlings and the height of tobacco plants aged 80- and 136-days were measured.

Ninety eight-day-old tobacco plants were used to compare the growth difference between the vector-transformed control and wild-type HMGS-OEs and mutant (S359A) HMGS-OEs. For each line, six (6) plants were used. The height of each plant, the fresh weight of four (4) leaves from the bottom of plants, the length of four (4) leaves from the bottom of plants and the width of four (4) leaves from the bottom of plants were recorded and analyzed respectively.

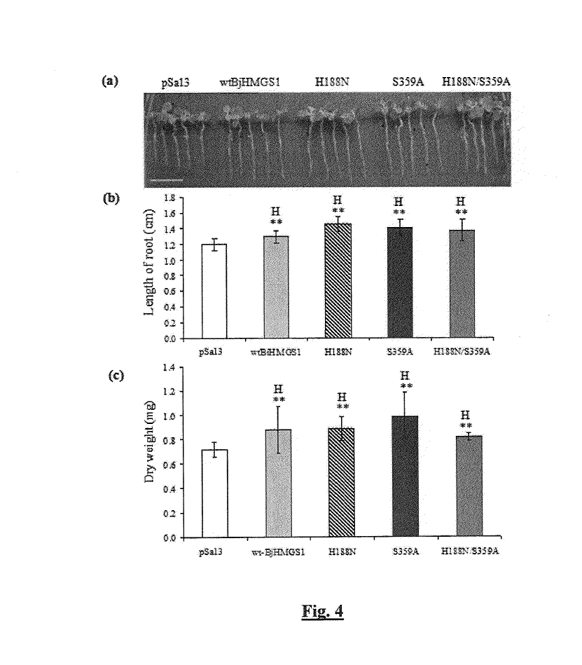

Phenotypic characters in growth were carried out on 14-day-old seedlings, 80-day-old plants and 136-day-old flowering plants of tobacco HMGS-OE. Both the root length (FIG. 4, panels (a) and (b)) and dry weight of tobacco HMGS-OE seedlings were significantly greater than the pSa13 vector-transformed controls, with OE-S359A showing the highest dry weight (FIG. 4, panel (c)).

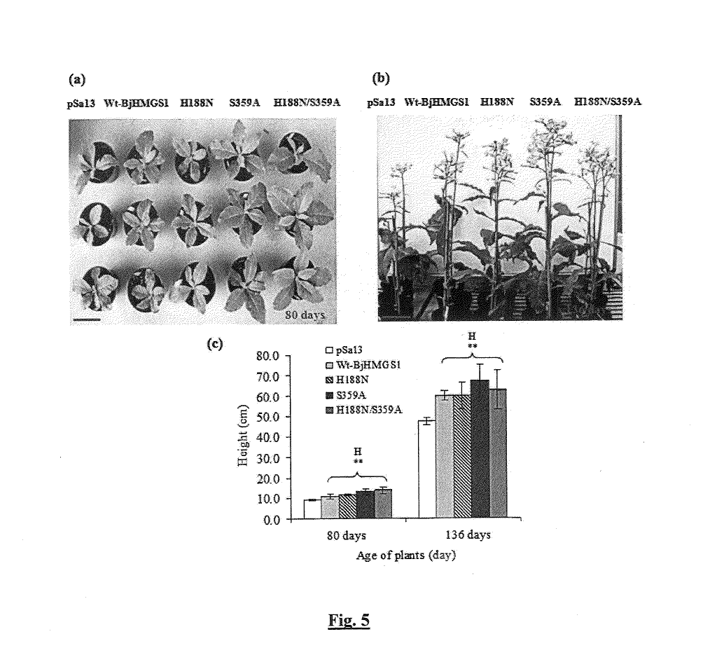

Both 80-day-old and 136-day-old tobacco OE plants grown in the greenhouse displayed greater height measurements than vector-transformed plants (FIG. 5). However, OE-H188N did not show as much increase as the OE-wtBjHMGS1 and OE-S359A (FIG. 5, panels (a) and (b)). This phenotype is consistent with the sterol change in different BjHMGS1-OEs and enzyme activities of the E. coli transformants expressing HMGS mutants in vitro (Nagegowda et al., Biochem. J. 383:517-527, (2004)).

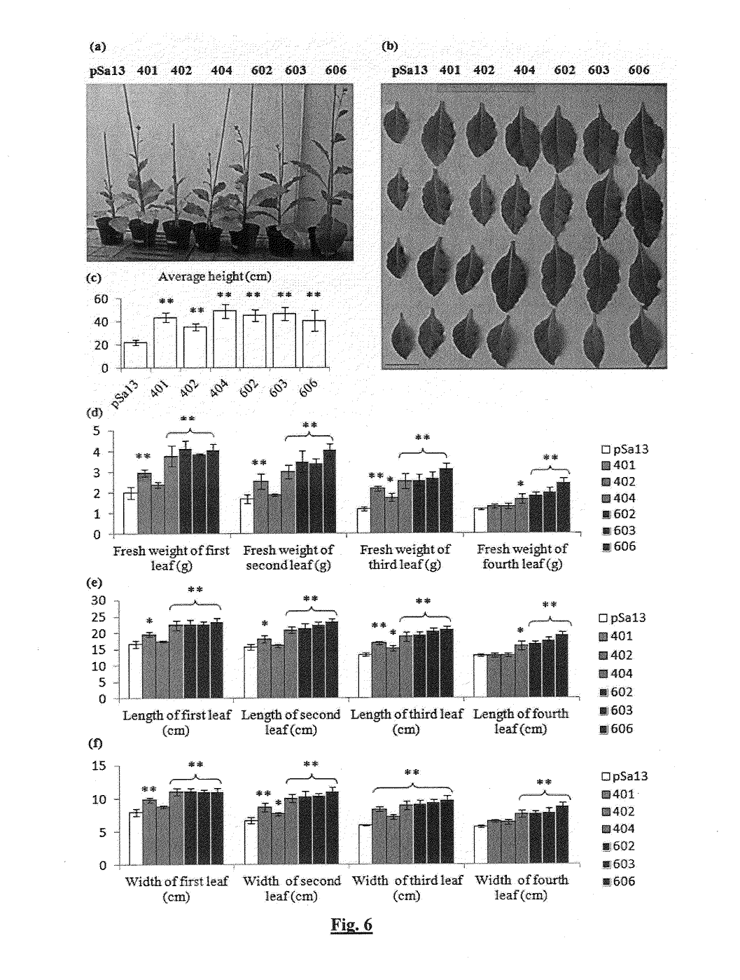

The growth difference between 98-day-old HMGS-OEs (OE-wtBjHMGS1 and OE-S359A) and 98-day-old vector-transformed control was also observed (FIG. 6). Both OE-wtBjHMGS1 and OE-S359A showed significantly higher (91% and 97% respectively) height than the vector-transformed control (FIG. 6, panel (c)). The fresh weight and leave size (length and width) in some of the OE-wtBjHMGS1 lines showed significantly heavier and bigger than the vector-transformed control of same age (FIG. 6, panels (d)-(f)). Furthermore, the fresh weight and leave size (length and width) in all of the mutant (S359A) HMGS-OE lines showed significantly heavier and bigger than the vector-transformed control of same age (FIG. 6, panels (d)-(f)).

### Example 7

### Tobacco HMGS-OEs Show Increased Seed Yield

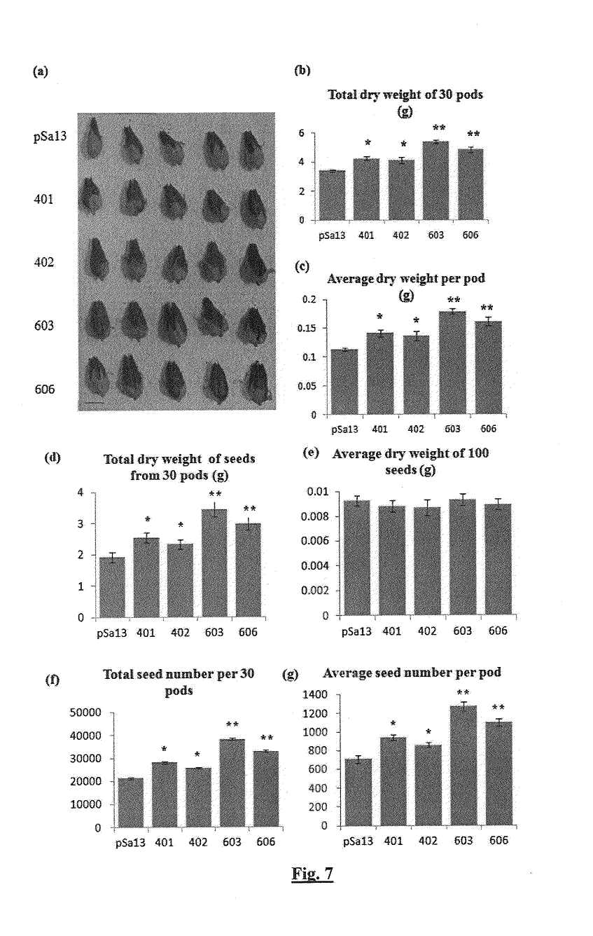

To test the differences in seed yield between vector (pSa13)-transformed tobacco and HMGS-OEs (OE-wtBjHMGS1 and OE-S359), two independent lines for each construct were used and 10 plants per line were grown. T₂ homozygous tobacco seeds of each line were first germinated on an MS plate. Two-week-old seedlings were transferred to soil. Tobacco pods were harvested when they were completely mature. The total dry pod weight, the average dry pod weight, total seed number in 30 pods and average seed number in each pod were recorded using 30 pods from each of 10 plants per line. The above procedure was repeated 3 times. It is shown that the HMGS-OEs exhibited increased seed yield in comparison to the vector-transformed control (FIG. 7). Seed yield in OE-wtBjHMGS1 increased 21 to 32% over the vector-transformed control (FIG. 7, panels (b)-(d)). Seed yield of OE-S359A showed 55 to 80% increase compare to the vector-transformed control (FIG. 7, panels (b)-(d)).

In order to further determine if seed size was altered in the HMGS-OEs, the dry weight of 100 tobacco seeds from each line was measured and 30 repeats were carried out for each line. The results showed that there was no significant difference between the vector-transformed control and HMGS-OEs in dry seed weight (of 100 seeds), suggesting that there is no significant difference in the seed size between the vector-transformed control and all the tested HMGS-OEs (FIG. 7, panel (e)). Therefore, HMGS-OE increase in seed yield is attributed to an increase in a tobacco pod size and seed number but not seed size (FIG. 7, panels (a)-(g)). It is noted that data shown in FIG. 7 are the average dry weight from 30 measurements of 100 tobacco seeds per line.

### Example 8

### Tobacco HMGS-OEs Regulates Expression of HMGS Downstream Genes

Nicotiana tabacum 3-hydroxy-3-methylglutaryl-CoA reductase (NtHMGR1 and NtHMGR2), isopentenyl-diphosphate delta-isomerase (NtIPI1 and NtIPI2), farnesyl diphosphate synthase (NtFPPS), squalene synthase (NtSQS), geranylgeranyl diphosphate synthase (NtGGPPS1), sterol methyltransferases (NtSMT1-2, NtSMT2-1 and NtSMT2-2) and cytochrome P450 monooxygenase (NtCYP85A1) are some of the downstream genes of HMGS encoding related intermediates in phytosterol and brassinosteroids (BR) biosynthesis. qRT-PCR was performed to check the effect of overexpression of BjHMGS1 on the expression of HMGS downstream genes in both seedlings and flowers of tobacco HMGS-OEs. In detail, total RNA (5 μg) of 20 day-old tobacco seedlings and fully-opened tobacco flowers were extracted using RNeasy Plant Mini Kit (Qiagen; catalog no. 74904) and were reverse transcribed into the first strand cDNA using the SuperScript First-Strand Synthesis System (Invitrogen; catalog no. 12371-019). Quantitative RT-PCR (qRT-PCR) was carried out with a StepOne Plus real-time PCR system (Applied Biosystems, Foster City, Calif., USA) and FastStart Universal SYBR Green Mater (Roche). The conditions for qRT-PCR were as follows: denaturation at 95° C. for 10 min, followed by 40 cycles of 95° C. for 15 s and 60° C. for 1 min. Three experimental replicates for each reaction were carried out using specific primers to the gene of interest and a tobacco actin as internal control. The relative changes of gene expression levels were analysed according to Schmittgen and Livak (Schmittgen and Livak, Nat. Protoc. 3:1101-1108, (2008)) from three independent experiments. Provided below are primers used for qRT-PCR analysis:

NtHMGR1-specific primers:
(SEQ ID NO: 11)
5′-TTGGCATCGGATTTGTTCAG-3′ (ML1879);
and
(SEQ ID NO: 12)
5′-GGCGGCTATCTTCCTCAAT-3′ (ML 1880);
NtHMGR2-specific primers:
(SEQ ID NO: 13)
5′-AGCAGGTGGCGTGAGAAAAT-3′ (ML1881);
(SEQ ID NO: 14)
5′-CGAACGACTGAACAAATCCG-3′ (ML1882);
NtIPI1-specific primers:
(SEQ ID NO: 15)
5′-ATCGGGTTTGTTCAGTCGTTC-3′ (ML1885);
(SEQ ID NO: 16)
5′-GCAGGTCCACGACGACTATCT-3′ (ML1886);
NtIPI2-specific primers:
(SEQ ID NO: 17)
5′-ATTGAGGAGAATGCTCTTGGTG-3′ (ML1887);
(SEQ ID NO: 18)
5′-CTGGTCAACTGGGACATCTTCA-3′ (ML1888);
NtFPPS-specific primers:
(SEQ ID NO: 19)
5′-CTTCTCCGCAACCACATCAC-3′ (ML1889);
(SEQ ID NO: 20)
5′-GAGGCAGTCTGGAACTCAACC-3′ (ML1890);
NtSQS-specific primers:
(SEQ ID NO: 21)
5′-AGGAGGTGGAAACAACTGATGA-3′ (ML1678);
(SEQ ID NO: 22)
5′-AGAACATACGGCACTTGGGT-3′ (ML1679);
NtGGPPS1-specific primers:
(SEQ ID NO: 23)
5′-GACCAATCCAACGCTATGCC-3′ (ML1891);
(SEQ ID NO: 24)
5′-TTATGGTTCGTCGGCTTTCC-3′ (ML1892);
NtSMT1-2-specific primers:
(SEQ ID NO: 25)
5′-TCCAAATAACGAAGAGCACGAA-3′ (ML1893);
(SEQ ID NO: 26)
5′-CAGGCGAATCCTCAGCAAG-3′ (ML1894);
NtSMT2-1-specific primers:
(SEQ ID NO: 27)
5′-AAACCGCCGATAAAGTCCC-3′ (ML1895);
(SEQ ID NO: 28)
5′-TCTACAGCCATTTCTTCGTGAA-3′ (ML1896);
NtSMT2-2-specific primers:
(SEQ ID NO: 29)
5′-ATCCACGGTATTGAAAGGGG-3′ (ML1897);
(SEQ ID NO: 30)
5′-TTTCAGCCTCGTCCACCAC-3′ (ML1898);
NtCYP85A1-specific primers:
(SEQ ID NO: 31)
5′-AAGAAAAGCCTTCCTCCTGGTA-3′ (ML1899);
(SEQ ID NO: 32)
5′-AAAACTCCCAAATCTGGCTCTT-3′ (ML1900);
NtACTIN-specific primers:
(SEQ ID NO: 33)
5′-TCACAGAAGCTCCTCCTAATCCA-3′ (ML1951);
(SEQ ID NO: 34)
5′-GAGGGAAAGAACAGCCTGAATG-3′ (ML1952).

Sequence data included herein can be found in the GenBank/EMBL data libraries under accession numbers U60452 (NtHMGR1), AF004232 (NtHMGR2), AB049815 (NtIPI1), AB049816 (NtIPI2), GQ410573 (NtFPPS), NTU60057 (NtSQS), EF382626 (NtGGPPS1), AF053766 (NtSMT1-2), U71108 (NtSMT2-1), U71107.1 (NtSMT2-2), DQ649022 (NtCYP85A1), U60489 (NtACTIN).

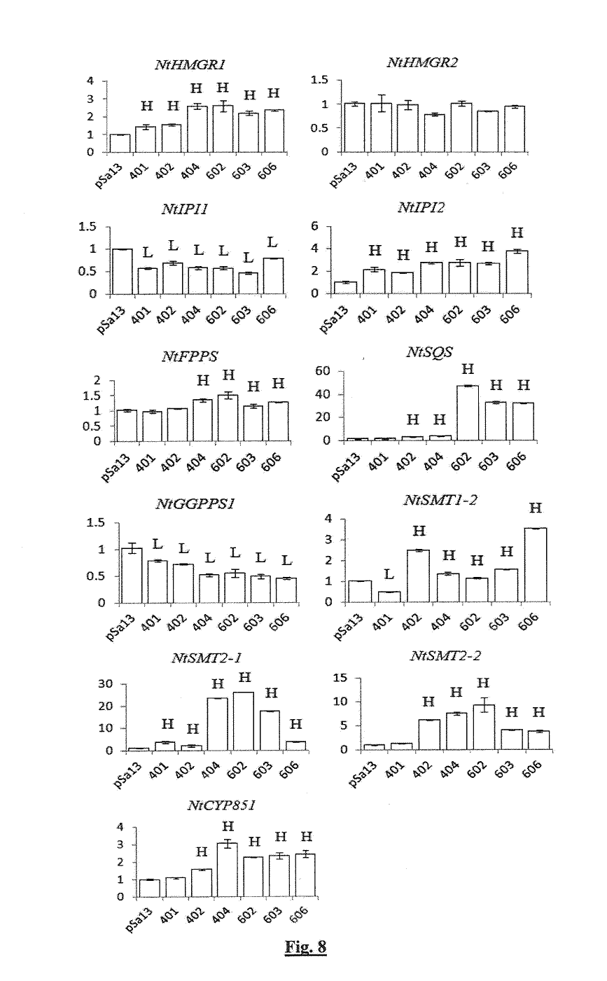

qRT-PCR results showed that the expression of NtHMGR1, NtIPI2, NtSQS, NtSMT1-2, NtSMT2-1, NtSMT2-2 and NtCYP85A1 were significantly higher than the vector-transformed control in both OE-wtBjHMGS1 and OE-S359A tobacco seedlings, while the expression of NtIPII and NtGGPPS1 were significantly lower than the vector-transformed control in both OE-wtBjHMGS1 and OE-S359A tobacco seedlings (P<0.05) (FIG. 8). Furthermore, there was no difference in the expression of NtHMGR2 between all the HMGS-OEs lines and the vector-transformed control in tobacco seedlings. For the expression of NtFPPS, there was no difference between two lines of OE-wtBjHMGS1 and the vector-transformed control, while the expression of NtFPPS in another OE-wtBjHMGS1 line and all the three OE-S359A lines was significantly higher than the vector-transformed control in tobacco seedlings (P<0.05) (FIG. 8).

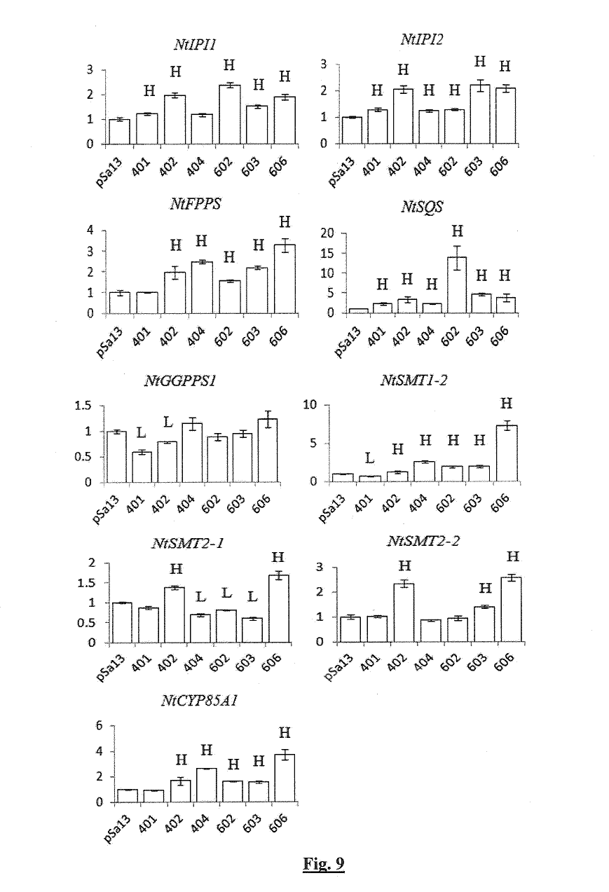

qRT-PCR results in tobacco flowers showed that the expression of NtHMGR1, NtIPI1, NtIPI2, NtFPPS, NtSQS, NtSMT1-2 and NtCYP85A1 were significantly higher than the vector-transformed control in both OE-wtBjHMGS1 and OE-S359A, while the expression of NtHMGR2 was significantly lower than the vector-transformed control (P<0.05) (FIG. 9). Furthermore, the expression of NtGGPPS1 didn't show up-regulation in both OE-wtBjHMGS1 and OE-S359A (FIG. 9).

In conclusion, in order to apply potential benefits of BjHMGS1 overexpression to plant distant from Brassica juncea, a model plant such as tobacco that has a more remote evolutionary relationship to Brassica juncea was selected for heterologous expression in this study. Wild-type and mutant BjHMGS1 (H188N, S359A and H188N/S359A) were overexpressed in tobacco (Nicotiana tabacum L. cv. Xanthi). Transgenic HMGS-overexpressor (OE)-S359A and OE-H188N/S359A displayed an increase in plant growth that was not obvious from previous analysis of transgenic Arabidopsis HMGS-OEs (Wang et al., Plant Biotechnol. J. 10:31-42, (2012)). Also, unlike transgenic Arabidopsis HMGS-OEs, tobacco overexpressing wild-type BjHMGS1 and mutant BjHMGS1 (OE-S359A) showed an increase (27% and 67%, respectively) in seed yield.

The above examples demonstrated that wild-type BjHMGS1 and mutant BjHMGS1, when overexpressed in tobacco, induced the expression of NtHMGR1 (FIGS. 1-2). The data also showed that transgenic tobacco overexpressing BjHMGS1 (HMGS-OEs) grows quicker, perhaps as a consequence of sterol accumulation in tobacco seedlings (FIGS. 3-6; Tables 1 and 2). Surprisingly, tobacco HMGS-OEs including OE-wtBjHMGS1 and OE-S359A showed 27% and 67% increase in seed yield, which may have arisen from the elevated sterol content in tobacco flowers (11.0-25.3%) (FIG. 7; Tables 3 and 4). Furthermore, the expression of some HMGS downstream genes, sterol intermediates biosynthetic genes and BR biosynthetic genes including NtHMGR1, NtIPI2, NtSQS, NtSMT1-2, NtSMT2-1, NtSMT2-2 and NtCYP85A1 were up-regulated, while the expression of NtIPI1 and NtGGPPS1 were down-regulated, in tobacco seedlings of OE-wtBjHMGS1 and OE-S359A (FIG. 8). The expression of NtHMGR1, NtIPI1, NtIPI2, NtFPPS, NtSQS, NtSMT1-2 and NtCYP85A1 were up-regulated, while the expression of NtHMGR2 was down-regulated, in tobacco flowers of OE-wtBjHMGS1 and OE-S359A (FIG. 9). Hence, HMGS is demonstrated herein to play an important role in plant growth and seed (grain) production by boosting seed yield besides increasing phytosterol content. It is anticipated that the growth and/or seed yield enhancement effect of overexpression of wild-type and mutant BjHMGS1 in tobacco can be extended to other plant species of the Solanaceae family. Provided herein is a new use of wild-type and mutant BjHMGS1 in enhancing plant growth and increasing seed yield, which would benefit food production in agriculture. Additionally, enhanced plant growth shortens time for harvest, which can be applied to forage, food crops, fiber crops, etc.

### Example 9

### Generation and Molecular Analyses of Transgenic Tomato HMGS-OEs

Wild-type tomato (Lycopersicon esculentum Mill. cv. UC82B, seeds obtained from Dr. WK Yip, The University of Hong Kong) was used in this study. Tomato plants were grown at 25° C. (16 hr light)/22° C. (8 hr in dark). Tomato seedlings were cultured in Murashige and Skoog medium (MS) (Murashige and Skoog, Physiol. Plant 15:473-497, 1962).

Agrobacterium tumefaciens-mediated transformation of tomato cotyledons and hypocotyls with A. tumefaciens LBA4404 harboring pAT332 was carried out following the procedures by Mathews et al. (The Plant Cell 15: 1689-1703, 2003) with minor modifications. Plasmids pBj134 (wtBjHMGS1) and pBj136 (S359A) were used in Agrobacterium-mediated transformation (Mathews et al., The Plant Cell 15: 1689-1703, 2003; Wang et al., Plant Biotechnol. J. 10:31-42, 2012). The binary vector pSa13 (Xiao et al., Plant Mol. Biol. 68: 574-583, 2008) was used as vector control in transformation. Tomato seeds were surface-sterilized in 75% ethanol for 1 min, and then rinsed three times in sterile water. The seeds were then soaked in 25% Clorox for 10 min and then rinsed four times with sterile water. The sterilized seeds were germinated on MS medium. Cotyledons and hypocotyls from 7-day-old seedlings were used as explants. The bacterial culture was grown overnight at 28° C. (OD₆₀₀ 0.5-0.6). Cells were harvested and suspended in liquid MS medium containing 0.2 mg/1 2,4-dichloro phenoxyacetic acid (2,4-D), 0.1 mg/1 indoleacetic acid (IAA) and 100 μM 3′,5′-dimethoxy-4′-hydroxyacetophenone (AS). The excised cotyledons and hypocotyls were incubated in the Agrobacterium suspension for 5 min with gentle shaking. Infected explants were co-cultivated on MS medium containing 2 mg/1 zeatin, 100 μM AS and 0.05 mg/1 IAA at 28° C. for 1 day in the dark and at 24° C. for 1 day with 16 hr light followed by washing twice with sterile water and once with liquid MS containing 2 mg/1 zeatin, 0.05 mg/1 IAA and 500 mg/1 carbencillin. Then they were transferred to selection medium (MS basal containing 2 mg/1 zeatin, 0.05 mg/1 IAA, 50 mg/1 kanamycin and 300 mg/1 carbencillin). One month after inoculation, the explants were sub-cultured on MS containing 1 mg/1 zeatin, 0.03 mg/1 IAA, 50 mg/1 kanamycin and 200 mg/1 carbencillin and subculture to fresh medium was carried out at least once a month. The regenerating shoots (4-5 cm high) were transferred to rooting medium (MS basal containing 0.1 mg/1 indole butyric acid (IBA), 50 mg/1 kanamycin and 200 mg/1 carbencillin) and the rooted seedlings were then transplanted to soil after acclimation.

T₁ transgenic tomato seeds were selected on MS containing kanamycin (50 μg/ml) and verified using PCR analysis with primers 35S and ML860, and DNA sequence analysis with primer ML915 following Wang et al. (Plant Biotechnol. J. 10:31-42, 2012). T₂ homozygous plants with a single-copy transgene were compared in plant growth.

### Example 10

### Western Blot and Southern Blot Analyses of Transgenic Tomato HMGS-OEs

Total protein was extracted (Chye et al., Plant J 18: 205-214, 1999) from 21-day-old tomato leaves. Protein concentration was determined using the Bio-Rad Protein Assay Kit I (Bio-Rad). Protein (20 μg per well) separated on 12% SDS-PAGE was transferred onto Hybond-ECL membrane (Amersham) using a Trans-Blot® cell (Bio-Rad). Antibodies raised against the synthetic peptide (DESYQSRDLEKVSQQ) (SEQ ID NO:5) corresponding to BjHMGS1 amino acids 290 to 304 were used in western blot analyses (Wang et al., Plant Biotechnol. J. 10:31-42, 2012) following Xiao et al. (The Plant Cell 22:1463-1482, 2010). Cross-reacting bands were detected using the ECL™ Western Blotting Detection Kit (Amersham) following the manufacturer's instructions.

Genomic DNA (40 μg) from 4-week-old tomato leaves prepared by the cetyltrimethylammonium bromide (CTAB) method (Rogers and Bendich, Plant Mol. Biol. 5: 69-76, 1985) was digested by EcoRI and separated on 0.7% agarose gel by electrophoresis, together with a 1-kb plus DNA standard ladder (Invitrogen). DNA was then transferred from the agarose gel onto Hybond-N membrane (Amersham) by capillary transfer (Southern, Nat Protoc 1: 518-525, 2006). Southern blot analysis of tomato using a digoxigenin-labelled full-length of BjHMGS1 cDNA probe with primer pair ML264 and ML276 was performed according to Wang et al. (Plant Biotechnol. J. 10:31-42, 2012). Primers are listed in Table 5.

TABLE 5
Oligonucleotide primers used in this study
(Bam HI restriction sites
in ML264 and ML276 are underlined)

| Gene | Primer | | Orien- | |
|---|---|---|---|---|
| Name | Name | Analysis | tation | Sequence (5′-3′) |
| CaMV | 35S | PCR | Forward | CAATCCCACTATCCTT |
| 35S | | | | CGCAAGACC |
| promoter | | | | (SEQ ID NO: 2) |
| BjHMGS1 | ML264 | Southern | Reverse | GGATCCATAACCAATG |
| | | Blot | | GACACTGAGGATCC |
| | | | | (SEQ ID NO: 3) |
| BjHMGS1 | ML915 | PCR | Forward | CATTGCTATGTTGATA |
| | | | | GGAC |
| | | | | (SEQ ID NO: 4) |
| BjHMGS1 | ML276 | Southern | Forward | GGATCCATGGCGAAGA |
| | | Blot | | ACGTAGGGATATTG |
| | | | | (SEQ ID NO: 7) |
| BjHMGS1 | ML860 | PCR | Reverse | GGAGACTGGTTCTCGC |
| | | | | AGAGAC |
| | | | | (SEQ ID NO: 8) |

### Example 11

### Growth Rate Measurement

Growth rate was measured according to previous reports (Johnston and Dore, Plant Physiol. 4: 31-62, 1929). T₂ homozygous plants with a single-copy transgene were compared in plant growth. Four-day-old tomato seedlings were transferred onto fresh MS plates placed vertically for a further 8-day growth. Twelve-day-old tomato seedlings of similar size were transferred from MS medium to soil for further growth rate measurements. Height measurements of 35-day-old and 63-day-old tomato plants were recorded. Two independent lines from each overexpression constructs were analysed. Thirty plants were used for measurement of height per individual line.

### Example 12

### PCR Analysis of Transgenic Tomato Lines and DNA Sequence Analyses to Confirm Presence of Transgenes

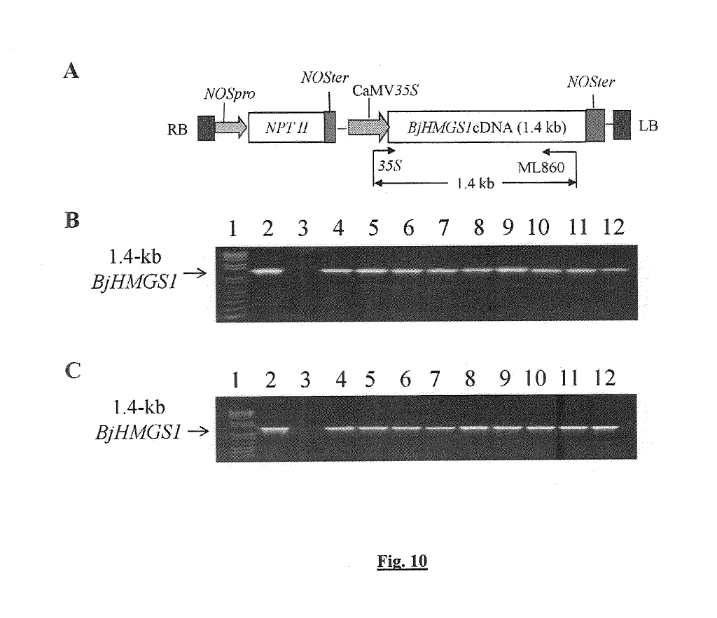

Putative tomato HMGS-OEs were designated as OE-wtBjHMGS1 (lines “401”, “403” and “404” etc.) and OE-S359A (lines “605”, “607” and “608” etc.). Transgenic tomato transformed with plasmids pBj134 (wtBjHMGS1) and pBj136 (S359A) was confirmed by PCR using the 35S promoter forward primer and a BjHMGS1 3′ cDNA reverse primer ML860 (Table 1) to amplify inserts of transgenes (FIG. 10, panel (a)). An expected 1.4-kb band was amplified from transgenic tomato lines (FIG. 10, panel (b) and panel (c)). In summary, 29 putative wild-type BjHMGS1 transgenic tomato lines were identified among 46 independent tomato lines tested in PCR analysis (FIG. 10, panel (b)) and 25 putative mutant BjHMGS1 (S359A) transgenic tomato lines were identified among 37 independent tomato lines tested in PCR analysis (FIG. 10, panel (c)). DNA sequence analysis was conducted on each PCR product amplified from wild-type and mutant BjHMGS1 transgenic tomato to verify wild-type BjHMGS1 and its mutant (S359A) sequences in each BjHMGS1 transformed line.

### Example 13

### Western Blot Analysis on PCR-Positive Wild-Type and Mutant (S359A) BjHMGS1 Transgenic Tomato Lines

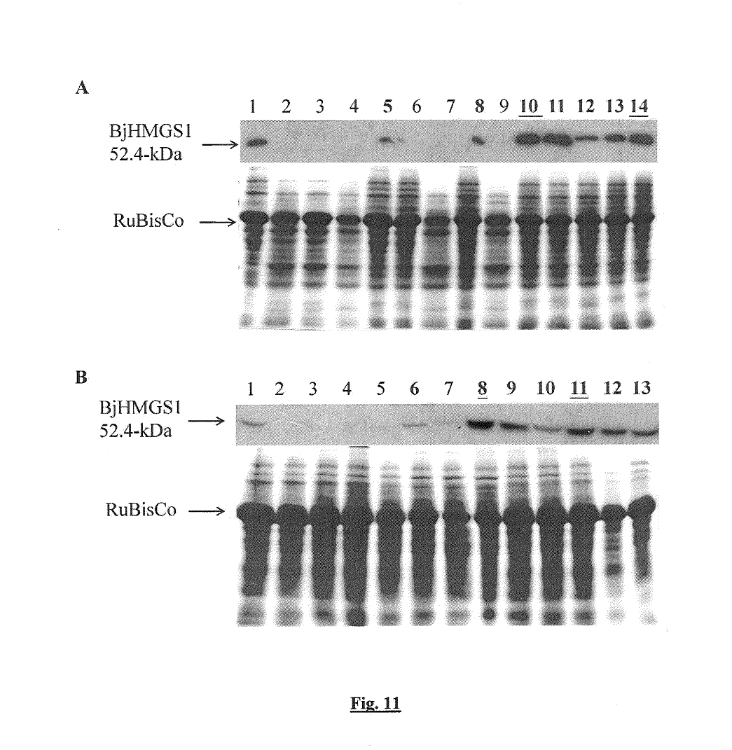

Western blot analysis was performed to further check if BjHMGS1 (52.4 kDa) was expressed in tomato transgenic plants by using antibodies against HMGS. Results verified that 17 of 29 PCR-positive wild-type BjHMGS1 transgenic tomato lines tested were confirmed to overexpress HMGS (FIG. 11, panel (a)). Additionally, 15 of 25 PCR-positive OE-S359A tomato lines tested were further confirmed to overexpress HMGS (FIG. 11, panel (b)).

### Example 14

### Southern Blot Analysis of EcoRI Digested DNA

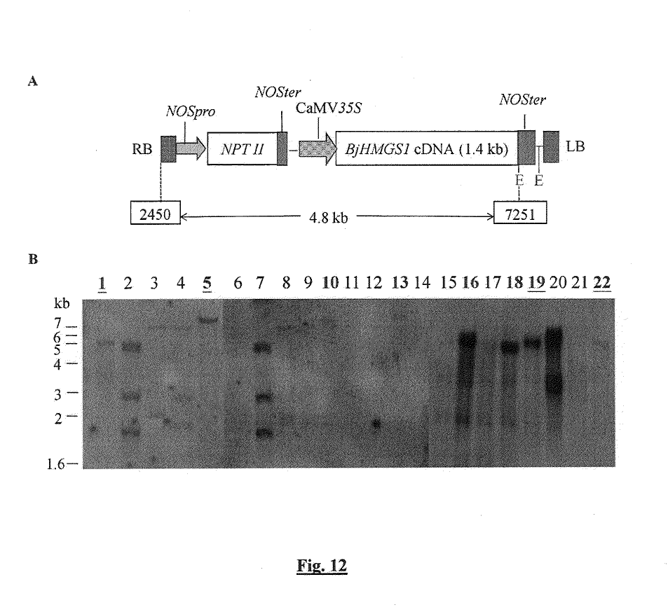

Southern blot analyses were performed to identify independent tomato HMGS-OE lines and check the copy number of transgenes in tomato HMGS-OEs. Results showed that four (4) lines each of OE-wtBjHMGS1 and OE-S359A had single-copy inserts of transgenes in Southern blot analysis (FIG. 12). Two such independent lines of OE-wtBjHMGS1 (“430” and “445”) and OE-S359A lines (“622” and “625”) were chosen for subsequent experiments.

### Example 15

### Tomato HMGS-OE Plants Show Increased Growth

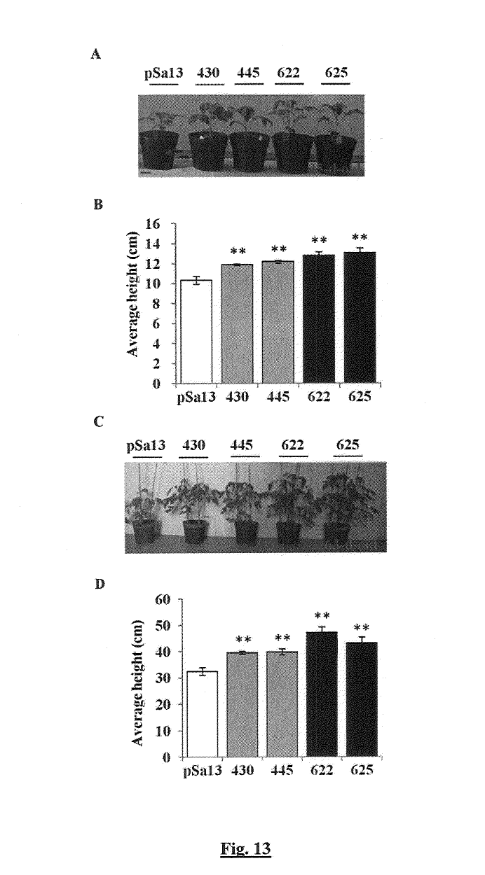

In order to check if the overexpression of BjHMGS1 has a similar effect in a crop plant (e.g., enhanced growth as displayed in tobacco HMGS-OEs), tomato was transformed by Agrobacterium-mediated transformation using plasmids pBj134 (wtBjHMGS1), pBj136 (S359A) and pSa13 (as control). Plasmids pBj134, pBj136 and pSa13 have been previously described (Wang et al., Plant Biotechnol. J. 10:31-42, 2012; Xiao et al., Plant Mol. Biol. 68: 574-583, 2008). Measurements of height to document growth differences between tomato HMGS-OEs (OE-wtBjHMGS1 and OE-S359A) and vector (pSa13)-transformed plants were carried out on 35-day-old and 63-day-old plants. Transgenic 35-day-old OE-wtBjHMGS1 and OE-S359A tomato plants showed significant increases (17% and 26%, respectively) in height over the vector-transformed control (FIG. 13, panel (a) and panel (b)). Consistently, growth differences in height between 63-day-old HMGS-OEs (OE-wtBjHMGS1 and OE-S359A) and vector-transformed plants were also evident (FIG. 13, panel (c)). Tomato OE-wtBjHMGS1 showed a significant increase (22%) in height over the control, while OE-S359A displayed an even greater increase (39%) in height over the control (FIG. 13, panel (d)).

In conclusion, the results demonstrate that the overexpression of OE-wtBjHMGS1 and OE-S359A caused enhanced growth in both transgenic tobacco and tomato plants. The realization of this strategy in improving growth in a crop such as tomato suggests that the method disclosed herein can be further extended to other economical/crop plants. The effect of the mutant BjHMGS1 construct OE-S359A was particularly significant in growth enhancement as 39% increase in height was observed over the control in 63-day-old transgenic tomato plant.

Having illustrated and described the principles of the present invention, it should be apparent to persons skilled in the art that the invention can be modified in arrangement and detail without departing from such principles. Those skilled in the art will recognize, or be able to ascertain using no more than routine experimentation, many equivalents to the specific embodiments of the invention described herein. Such equivalents are intended to be encompassed by the following claims.

All publications and published patent documents cited in this specification are incorporated herein by reference to the same extent as if each individual publication or patent application was specifically and individually indicated to be incorporated by reference.

We claim:

1. A transgenic plant genetically engineered to overexpress one or more exogenous 3-hydroxy-3-methylglutaryl-CoA synthase 1 (HMGS1) in an amount effective to enhance growth and/or seed yield relative to a control plant, wherein said transgenic plant belongs to the Solanaceae family, and said one or more exogenous HMGS1 comprises an amino acid sequence at least 77% identical to SEQ ID NO:6.

2. The transgenic plant of claim 1, wherein said transgenic plant comprises an exogenous nucleic acid sequence encoding a HMGS1, said HMGS1 comprising an amino acid sequence as set forth in SEQ ID NO:6 except that the amino acid residue serine at position 359 is changed to amino acid residue alanine.

3. The transgenic plant of claim 1, wherein said transgenic plant comprises an exogenous nucleic acid sequence encoding a HMGS1, said HMGS1 comprising an amino acid sequence as set forth in SEQ ID NO:6 except that the amino acid residue histidine at position 188 is changed to amino acid residue asparagine and the amino acid residue serine at position 359 is changed to amino acid residue alanine.

4. The transgenic plant of claim 1, wherein said transgenic plant comprises an exogenous nucleic acid sequence encoding a HMGS1, said HMGS1 comprises an amino acid sequence as set forth in SEQ ID NO:6.

5. The transgenic plant of claim 1, wherein said transgenic plant is selected from the group consisting of tobacco, potato, tomato, pepper, and eggplant.

6. The transgenic plant of claim 5, wherein said transgenic plant is tobacco or tomato.

7. A seed from the transgenic plant of claim 1, said seed overexpressing said one or more exogenous HMGS1.

8. A progeny of the transgenic plant of claim 1, said progeny overexpressing said one or more exogenous HMGS1.

9. A plant product derived from the transgenic plant of claim 1, said plant product overexpressing said one or more exogenous HMGS1.

10. A method of enhancing plant growth and/or seed yield, comprising genetically engineering a plant to overexpress one or more exogenous 3-hydroxy-3-methylglutaryl-CoA synthase 1 (HMGS1) in an amount effective to enhance growth and/or seed yield relative to a control plant, wherein said one or more exogenous HMGS1 comprise an amino acid sequence at least 77% identical to SEQ ID NO:6.

11. The method of claim 10, comprising:
(a) transforming a plant with a vector comprising one or more exogenous nucleic acid sequences encoding said one or more exogenous HMGS1 operably linked to one or more plant expressible promoter; and
(b) expressing said one or more exogenous HMGS1 in said plant in an amount effective to provide enhanced growth and/or seed yield relative to a control plant.

12. The method of claim 11, wherein said vector comprises an exogenous nucleic acid sequence encoding a HMGS1, said HMGS1 comprising an amino acid sequence as set forth in SEQ ID NO:6 except that the amino acid residue serine at position 359 is changed to amino acid residue alanine.

13. The method of claim 11, wherein said vector comprises an exogenous nucleic acid sequence encoding a HMGS1, said HMGS1 comprising an amino acid sequence as set forth in SEQ ID NO:6 except that the amino acid residue histidine at position 188 is changed to amino acid residue asparagine and the amino acid residue serine at position 359 is changed to amino acid residue alanine.

14. The method of claim 10, wherein said vector comprises an exogenous nucleic acid sequence encoding a HMGS1, said HMGS1 comprising an amino acid sequence as set forth in SEQ ID NO:6.

15. The method of claim 10, wherein said plant belongs to the Solanaceae family.

16. The method of claim 15, wherein said plant is selected from the group consisting of tobacco, potato, tomato, pepper, and eggplant.

17. The method of claim 16, wherein said plant is tobacco or tomato.

18. The method of claim 11, wherein said one or more plant expressible promoter is selected from the group consisting of a constitutive promoter, a tissue-specific promoter and an inducible promoter.

19. A method of screening for a functional variant of Brassica juncea HMGS1, said Brassica juncea HMGS1 comprising the amino acid sequence as set forth in SEQ ID NO:6, comprising:
(a) obtaining a plant cell genetically modified to express a candidate variant;
(b) regenerating a pant from said plant cell; and
(c) determining whether said plant exhibits an increase in growth and/or seed yield, thereby determining whether the candidate variant is a functional equivalent of said Brassica juncea HMGS1.

20. The method of claim 19, wherein said plant cell belongs to the Solanaceae family.
